(19) United States
(12) Patent Application Publication
(10) Pub. No.: US 2010/0190710 A1
(43) Pub. Date: Jul. 29, 2010

(54) IL-23 RECEPTOR ANTAGONISTS AND USES THEREOF

(75) Inventors: Sylvain Chemtob, Cote-St Luc (CA); Christiane Quiniou, Montreal (CA); William D. Lubell, Montreal (CA)

Correspondence Address: CHOATE, HALL & STEWART LLP, TWO INTERNATIONAL PLACE, BOSTON, MA, 02110, US

(73) Assignee: Valorisation HSJ, Societe en Commandite, QUEBEC (CA)

(21) Appl. No.: 12/666,028

(22) PCT Filed: Jul. 7, 2008
(86) PCT No.: PCT/IB08/02490

§ 371 Date: Apr. 8, 2010

Related U.S. Application Data
(60) Provisional application No. 60/958,660, filed on Jul. 6, 2007.

(51) Int. Cl. A61K 38/16; C07K 14/00; C07K 7/08; C07K 7/06; C07K 5/10; C07K 5/08; C12N 15/63; C12N 5/10; A61K 38/10; A61K 38/08; A61K 38/05; A61K 38/06; A61K 38/07; A61P 29/00; A61P 37/00; G01N 33/53

(52) U.S. Cl. 514/12; 530/324; 530/325; 530/326; 530/327; 530/328; 530/329; 530/330; 530/331; 435/320.1; 435/325; 514/13; 514/14; 514/15; 514/16; 514/17; 514/18; 514/19; 435/7.2

(57) ABSTRACT

The present invention relates to IL-23 receptor antagonists and agonists. The use of IL-23 receptor antagonists in treating autoimmune and inflammatory disorders, as well as methods of identifying IL-23 receptor antagonists and agonists.

### CROSS-REFERENCE TO RELATED APPLICATIONS

The present application claims benefit of the filing date of U.S. Provisional Application Ser. No. 60/958,660, filed Jul. 6, 2007, the disclosure of which is incorporated herein in its entirety.

### FIELD OF THE INVENTION

The present invention relates to IL-23 receptor antagonists and agonists, their use, and methods of identifying such antagonists and agonists.

### BACKGROUND OF THE INVENTION

Autoimmunity (and inflammation) underlie a variety of human afflictions such as inflammatory bowel disease (IBD), psoriasis, and multiple sclerosis. At present, over 30-50% of patients with IBD, psoriasis, and multiple sclerosis fail to respond to traditional and current disease modifying anti-rheumatic drugs (DMARDs) including new biologics (e.g., anti-TNF antibodies).

The cytokine interleukin (IL)-23 plays a pivotal role in the establishment and maintenance of inflammatory autoimmune diseases and has emerged as the key player in IBD, psoriasis, and multiple sclerosis. Compelling human genetic evidence strongly points to a role for IL-23 in these diseases (Alifirova et al., Zh Nevrol Psikhiatr Im S S Korsakova Spec No 3:130-135 (2006); Cargill et al., Am J Hum Genet. 80:273-290 (2007); Duerr, et al., Science 314:1461-1463 (2006); Seegers et al., Genes Immun 3:419-423 (2002); Zwiers et al., Genes Immun 5:675-677 (2004)) and mechanistic (Gocke et al., J Immunol 178:1341-1348 (2007); Hunter, Nat Rev Immunol, 5:521-531 (2005); Langrish et al., Immunol Rev 202:96-105 (2004); Monteleone et al., Curr Opin Gastro 22:361-364 (2006)). Increased levels of IL-23 are observed in intestinal tissue from patients with IBD (Stallmach et al., Intl J Colorectal Dis 19:308-315 (2004)), in psoriatic dermal lesions (Lee et al., J Exp Med 199:125-130 (2004)), and in plaques of multiple sclerosis (Li et al., Brain 130:490-501 (2007)). A variety of strategies have validated IL-23 as an important target in mentioned autoimmune diseases, including (animal) gene disruption (Hunter, Nat Rev Immunol, 5:521-531 (2005), antibodies against its p40 subunit (Chen et al., J Clin Invest 116:1317-1326 (2006); Kasper et al., Curr Med Res Opin 22:1671-1678 (2006); Krueger et al, N Engl J Med 356:580-592 (2007); Mannon et al., N Engl J Med 351:2069-2079 (2004)), and small inhibitors of release of IL-23 (and IL-12) (Burakoff et al., Inflamm Bowel Dis 12:558-565 (2006)).

IL-23 belongs to the IL-12 family of cytokines; these cytokines are structurally related (Hunter, Nat Rev Immunol, 5:521-531 (2005)). IL-23 is produced by T cells and mostly macrophages, and acts on the transmembrane IL-23 receptor (IL-23R). IL-23R is predominantly present on memory CD4⁺ T helper cells (T_{H}17) required for the induction and maintenance of chronic inflammation. The effects of IL-23 are largely mediated through IL-17 (Hunter, Nat Rev Immunol, 5:521-531 (2005)). IL-23 is composed of IL-23p19 and IL-12p40 subunits, and shares the latter with IL-12. Accordingly, the role played by IL-12p40 in autoimmune inflammation has long been misinterpreted to be attributed to IL-12 until recent studies clearly revealed that it is IL-23, and not IL-12, that is the decisive factor in this immune deviation (Cua et al., Nature 421:744-748 (2003); Holscher, Curr Opin Investig Drugs 6:489-495 (2005); Kreymborg et al., Expert Opin Ther Targets 9:1123-1136 (2005)).

The failure of current therapies highlights the fact that effective treatment for autoimmune disease remains a serious unmet medical need. There remains a need to develop new therapeutic strategies to block the inflammatory process and to avoid the side effects of pharmacologic treatments.

### SUMMARY OF THE INVENTION

The present invention features IL-23 receptor antagonists and agonists. The invention also features methods of treating autoimmune and inflammatory disorders using the IL-23 receptor antagonists of the invention, as well as methods of identifying additional IL-23 receptor antagonists and agonists.

Accordingly, in the first aspect, the invention features a compound comprising a sequence (e.g., an amino acid sequence) characterized by the formula T₁E₂E₃E₄Q₅Q₆Y₇L₈, where the compound is 25 or fewer amino acids in length, where the compound antagonizes a biological activity of an interleukin 23 (IL-23) receptor, and where;

T₁ is no residue, threonine, phenylalanine, alanine, or Σ, where Σ defines an alpha-amino acid comprising a hydrophobic side-chain Σ or aromatic side chain;

E₂, E₃, and E₄ each independently is no residue, alanine, glutamic acid, glutamine, aspartic acid, asparagine, serine, histidine, homoserine, beta-leucine, beta-phenylalanine, alpha amino adipic acid, or Ψ where Ψ is a 3-amino-5-phenylpentanoic acid-alpha-amino acid comprising a hydrophobic side-chain, an aromatic amine, an aliphatic amine or a primary arylalkyl amine;

Q₅ and Q₆ each independently is no residue, alanine, glutamine, aspartic acid, asparagine, glutamic acid, serine, homoserine, alpha-amino adipic acid, or ψ where ψ is a 3-amino-5-phenylpentanoic acid-alpha-amino acid comprising a hydrophobic side-chain, an aromatic amine, an aliphatic amine or a primary arylalkyl amine;

Y₇ is no residue, tyrosine, phenylalanine, tryptophan, alanine, histidine, pyridylalanine, or Σ where Σ is an alpha-amino acid comprising a hydrophobic side-chain Σ or aromatic side chain or heteroaromatic side chain; and

L₈ is no residue, leucine, alanine, valine, methionine, phenylalanine, tryptophan, or φ where φ is an alpha-amino acid comprising a hydrophobic side-chain; an aliphatic amine of one to ten carbons; or an aromatic or arylalkylamine; or a heteroaromatic or heteroarylalkylamine.

In a desirable embodiment of the first aspect of the invention, the compound comprises at least one D-amino acid. In other desirable embodiments, the compound comprises two, three, four, five, six, seven, or eight D-amino acids or consists entirely of D-amino acids. In another desirable embodiment, the compound is a reverse-D or reverse-L peptide or a reverse peptide containing a combination of L- and D-amino acids.

In additional desirable embodiments of the first aspect of the invention, the alpha-amino acid comprising a hydrophobic side-chain is nor-leucine, iso-leucine, tert-leucine, cyclohexylalanine, or allylglycine; the aliphatic amine of one to ten carbons is a methylamine, iso-butylamine, iso-valerylamine, cyclopentylamine, cyclohexylmethylamine or cyclohexylamine; and the aromatic or arylalkylamine is aniline, naphtylamine, benzylamine, cinnamylamine, or phenylethylamine; and the heteroaromatic or heteroarylalkylamine is pyridylamine, pyridylmethylamine, or tryptamine.

In further desirable embodiments of the first aspect of the invention, the alpha-amino acid comprising a hydrophobic side-chain Σ or aromatic side chain is nor-leucine, iso-leucine, tert-leucine, cyclohexylalanine, allylglycine, napthylalanine, pyridylalanine, histidine, tyrosine, alanine, valine, isoleucine, leucine, methionine, phenylalanine, tryptophan, or Λ where Λ is a neutral aliphatic amino acid; an aliphatic amine of one to ten carbons; an aromatic or arylalkylamine; tyrosine; 4-hydroxyphenylglycine; phenylglycine; homoserine; 3,4-dihydroxyphenylalanine; or 4-chlorophenylalanine. Desirably, the aliphatic amine of one to ten carbons is a methylamine, iso-butylamine, iso-valerylamine, cyclopentylamine, cyclohexylmethylamine, or cyclohexylamine; and the aromatic or arylalkylamine is aniline, naphtylamine, benzylamine, cinnamylamine, or phenylethylamine; and the heteroaromatic or heteroarylalkylamine is pyridylamine, pyridylmethylamine, or tryptamine.

In another desirable embodiment of the first aspect of the invention, the primary arylalkyl amine is a benzylamine, phenylethylamine, 2,2-diphenylethylamine, or 4-phenyl-benzylamine.

In other desirable embodiments of the first aspect of the invention, the compound consists of the sequence TEEEQQYL (SEQ ID NO:1), TEEAQQYL (SEQ ID NO:6), TAAEQQYL (SEQ ID NO:7), TAAAQQYL (SEQ ID NO:8), EEEQQYL (SEQ ID NO:9), EEQQYL (SEQ ID NO:10), EQQYL (SEQ ID NO:11), AEEQQYL (SEQ ID NO:12), TEEEQQY (SEQ ID NO:13), TEEEQQ (SEQ ID NO:14), TEEEQ (SEQ ID NO:15), TEEE (SEQ ID NO:16), TEEEQAYL (SEQ ID NO:17), or TEEEAAYL (SEQ ID NO:18), where, desirably, at least one amino acid is a D-amino acid.

In yet another desirable embodiment of the first aspect of the invention, the compound further comprises G₁ attached to the amino-terminus of the sequence, G₂ attached to the carboxy-terminus of the sequence, or G₁ attached to the amino-terminus of the sequence and G₂ attached to the carboxy-terminus of the sequence, where G₁ is no residue, a hydrogen, a straight chained or branched alkyl group of one to eight carbons, or an acyl group, and G₂ is no residue, a hydrogen, NH₂, an aliphatic amine of one to ten carbons, or an aromatic or arylalkyl amine, or heteroaromatic or heteroarylalkylamine. Desirably, the acyl group is an acetyl, propionyl, butanyl, iso-propionyl, or iso-butanyl; the aliphatic amine of one to ten carbons is a methylamine, iso-butylamine, iso-valerylamine, cyclopentylamine, cyclohexylmethylamine, or cyclohexylamine; and the aromatic or arylalkyl amine is aniline, napthylamine, benzylamine, cinnamylamine, or phenylethylamine, and the heteroaromatic or heteroarylalkylamine is pyridylamine, pyridylmethylamine, or tryptamine.

The second aspect of the invention features a compound comprising a sequence (e.g., an amino acid sequence) characterized by the formula K₁K₂Y₃L₄V₅W₆V₇Q₈, where the compound is 25 or fewer amino acids in length, where the compound antagonizes a biological activity of an interleukin 23 (IL-23) receptor, and where;

K₁ and K₂ each independently is no residue, lysine, arginine, histidine, alanine, ornithine, citruline, 2-pyridylalanine, 3-pyridylalanine, 4-pyridylalanine, or an arginine surrogate;

Y₃ is no residue, tyrosine, phenylalanine, tryptophan, alanine, or Σ where Σ is an alpha-amino acid comprising a hydrophobic side-chain Σ or aromatic side chain, or heteroaromatic side chain;

L₄ is no residue, leucine, alanine, valine, methionine, phenylalanine, tryptophan, or φ where φ is an alpha-amino acid comprising a hydrophobic side-chain; an aliphatic amine of one to ten carbons; or an aromatic or arylalkylamine; or a heteroaromatic or heteroarylalkylamine; or a heteroaromatic or heteroarylalkylamine;

V₅ is no residue, valine, leucine, alanine, methionine, phenylalanine, tryptophan, or 4) where 4) is an alpha-amino acid comprising a hydrophobic side-chain; an aliphatic amine of one to ten carbons; or an aromatic or arylalkylamine; or a heteroaromatic or heteroarylalkylamine;

W₆ is no residue, tryptophan, tyrosine, phenylalanine, alanine, or Σ where Σ is an alpha-amino acid comprising a hydrophobic side-chain Σ or aromatic side chain; or heteroaromatic side chain;

V₇ is no residue, valine, leucine, alanine, methionine, phenylalanine, tryptophan, or φ where φ is an alpha-amino acid comprising a hydrophobic side-chain; an aliphatic amine of one to ten carbons; or an aromatic or arylalkylamine; or a heteroaromatic or heteroarylalkylamine; and

Q₈ is no residue, glutamine, aspartic acid, asparagine, glutamic acid, serine, or ψ where ψ is a 3-amino-5-phenylpentanoic acid-alpha-amino acid comprising a hydrophobic side-chain, an aromatic amine, an aliphatic amine or a primary arylalkyl amine.

In a desirable embodiment of the second aspect of the invention, the compound comprises at least one D-amino acid. Desirably, the compound of the second aspect of the invention comprises two, three, four, five, six, seven, or even eight D-amino acids or consists entirely of D-amino acids. In another desirable embodiment, the compound is a reverse-D or reverse-L peptide or a reverse peptide containing a combination of L- and D-amino acids.

In other desirable embodiments of the second aspect of the invention, the alpha-amino acid comprising a hydrophobic side-chain is nor-leucine, iso-leucine, tert-leucine, cyclohexylalanine, or allylglycine; the aliphatic amine of one to ten carbons is a methylamine, iso-butylamine, iso-valerylamine, cyclopentylamine, cyclohexylmethylamine, or cyclohexylamine; and the aromatic or arylalkylamine is aniline, naphtylamine, benzylamine, cinnamylamine, or phenylethylamine; and the heteroaromatic or heteroarylalkylamine is pyridylamine, pyridylmethylamine, or tryptamine.

In additional desirable embodiments of the second aspect of the invention, the alpha-amino acid comprising a hydrophobic side-chain Σ or aromatic side chain is nor-leucine, iso-leucine, tert-leucine, cyclohexylalanine, allylglycine, napthylalanine, pyridylalanine, histidine, tyrosine, alanine, valine, isoleucine, leucine, methionine, phenylalanine, tryptophan, or Λ where Λ is a neutral aliphatic amino acid; an aliphatic amine of one to ten carbons; an aromatic or arylalkylamine; tyrosine; 4-hydroxyphenylglycine; phenylglycine; homoserine; 3,4-dihydroxyphenylalanine; or 4-chlorophenylalanine.

In further desirable embodiments of the second aspect of the invention, the aliphatic amine of one to ten carbons is a methylamine, iso-butylamine, iso-valerylamine, cyclopentylamine, cyclohexylmethylamine, or cyclohexylamine; the aromatic or arylalkylamine is aniline, naphtylamine, benzylamine, cinnamylamine, or phenylethylamine; the heteroaromatic or heteroarylalkylamine is pyridylamine, pyridylmethylamine, or tryptamine; the arginine surrogate is 4-amidinophenylacetyl, 4-amidinophenylpropionyl, 4-amidinophenylglycyl, 4-amidinophenylmethylglycyl, 4-guanidinophenylacetyl, 4-uanidinophenylpropionyl, 4-guanidinophenylglycyl, or 4-guanidinophenylmethylglycyl; and the primary arylalkyl amine is a benzylamine, phenylethylamine, 2,2-diphenylethylamine, or 4-phenyl-benzylamine.

In yet other desirable embodiments of the second aspect of the invention, the compound further comprises G₁ attached to the amino-terminus of the sequence, G₂ attached to the carboxy-terminus of the sequence, or G₁ attached to the amino-terminus of the sequence and G₂ attached to the carboxy-terminus of the sequence, where G₁ is no residue, a hydrogen, a straight chained or branched alkyl group of one to eight carbons, or an acyl group, and G₂ is no residue, a hydrogen, NH₂, an aliphatic amine of one to ten carbons, or an aromatic or arylalkyl amine. Desirably, the acyl group is an acetyl, propionyl, butanyl, iso-propionyl, or iso-butanyl; the aliphatic amine of one to ten carbons is a methylamine, iso-butylamine, iso-valerylamine, cyclopentylamine, cyclohexylmethylamine, or cyclohexylamine; and the aromatic or arylalkyl amine is aniline, napthylamine, benzylamine, cinnamylamine, or phenylethylamine; and the heteroaromatic or heteroarylalkylamine is pyridylamine, pyridylmethylamine, or tryptamine.

The third aspect of the invention features a compound comprising a sequence (e.g., an amino acid sequence) characterized by the formula M₁E₂E₃S₄K₅Q₆L₇Q₈L₉, where the compound is 25 or fewer amino acids in length, where the compound antagonizes a biological activity of an interleukin 23 (IL-23) receptor, and where;

M₁ is no residue, methionine, valine, leucine, alanine, phenylalanine, tryptophan, or φ where φ is an alpha-amino acid comprising a hydrophobic side-chain; an aliphatic amine of one to ten carbons; or an aromatic or arylalkylamine;

E₂ and E₃ each independently is no residue, alanine, glutamic acid, glutamine, aspartic acid, asparagine, serine, histidine, homoserine, beta-leucine, beta-phenylalanine, alpha amino adipic acid, trimesic acid, or an alpha,omega-dicarboxylic acid (e.g., succinic acid, glutaric acid, or azelic acid) or Ψ where Ψ is a 3-amino-5-phenylpentanoic acid-alpha-amino acid comprising a hydrophobic side-chain, an aromatic amine, an aliphatic amine or a primary arylalkyl amine;

S₄ is no residue, serine, threonine, allothreonine, hydroxyproline, beta-hydroxyvaline, valine, or η where η is a neutral hydrophilic amino acid;

K₅ is no residue, glutamine, lysine, arginine, histidine, alanine, ornithine, citruline, 2-pyridylalanine, 3-pyridylalanine, 4-pyridylalanine, or an arginine surrogate;

Q₆ is no residue, alanine, glutamine, aspartic acid, asparagine, glutamic acid, serine, or ψ where ψ is a 3-amino-5-phenylpentanoic acid-alpha-amino acid comprising a hydrophobic side-chain, an aromatic amine, an aliphatic amine or a primary arylalkyl amine;

L₇ is no residue, leucine, alanine, valine, methionine, phenylalanine, tryptophan, or φ where φ is an alpha-amino acid comprising a hydrophobic side-chain; an aliphatic amine of one to ten carbons; or an aromatic or arylalkylamine; or a heteroaromatic or heteroarylalkylamine;

Q₈ is no residue, glutamine, aspartic acid, asparagine, glutamic acid, serine, or ψ where ψ is a 3-amino-5-phenylpentanoic acid-alpha-amino acid comprising a hydrophobic side-chain, an aromatic amine, an aliphatic amine or a primary arylalkyl amine; and

L₉ is no residue, leucine, isoleucine, alanine, valine, methionine, phenylalanine, tryptophan, or φ where φ is an alpha-amino acid comprising a hydrophobic side-chain; an aliphatic amine of one to ten carbons; or an aromatic or arylalkylamine; or a heteroaromatic or heteroarylalkylamine.

In a desirable embodiment of the third aspect of the invention, the compound comprises at least one D-amino acid. Desirably, the compound of the third aspect of the invention comprises two, three, four, five, six, seven, eight, or even nine D-amino acids or consists entirely of D-amino acids. In another desirable embodiment, the compound is a reverse-D or reverse-L peptide or a reverse peptide containing a combination of L- and D-amino acids.

In other desirable embodiments of the third aspect of the invention, the neutral amino acid is hydroxyvaline, beta,beta-dialkylserine, homo-serine, allothreonine, or hydroxyproline; the alpha-amino acid comprising a hydrophobic side-chain is nor-leucine, iso-leucine, tert-leucine, cyclohexylalanine, or allylglycine; the aliphatic amine of one to ten carbons is a methylamine, iso-butylamine, iso-valerylamine, cyclopentylamine, cyclohexylmethylamine, or cyclohexylamine; and the aromatic or arylalkylamine is aniline, naphtylamine, benzylamine, cinnamylamine, or phenylethylamine; and the heteroaromatic or heteroarylalkylamine is pyridylamine, pyridylmethylamine, or tryptamine; the arginine surrogate is 4-amidinophenylacetyl, 4-amidinophenylpropionyl, 4-amidinophenylglycyl, 4-amidinophenylmethylglycyl, 4-guanidinophenylacetyl, 4-iianidinophenylpropionyl, 4-guanidinophenylglycyl, or 4-guanidinophenylmethylglycyl; and the primary arylalkyl amine is a benzylamine, phenylethylamine, 2,2-diphenylethylamine, or 4-phenyl-benzylamine.

In other desirable embodiments of the third aspect of the invention, the compound consists of the sequence MEESKQLQL (SEQ ID NO:2), MAESKQLQL (SEQ ID NO:19), MAASKQLQL (SEQ ID NO:20), ESKQLQL (SEQ ID NO:21), MEESKQLQI (SEQ ID NO:22), MEESKQL (SEQ ID NO:23), MEESKQ (SEQ ID NO:24), MEESQQLQI (SEQ ID NO:25), EESKQLQL (SEQ ID NO:26), VQAANALGMEESKQLQLHLDDLVL (SEQ ID NO:27), or LVLDDLHLQLQKSEEMGLANAAQV (SEQ ID NO:28), where, desirably, at least one amino acid is a D-amino acid.

In additional embodiments of the third aspect of the invention, the compound further comprises G₁ attached to the amino-terminus of the sequence, G₂ attached to the carboxy-terminus of the sequence, or G₁ attached to the amino-terminus of the sequence and G₂ attached to the carboxy-terminus of the sequence, where G₁ is no residue, a hydrogen, a straight chained or branched alkyl group of one to eight carbons, or an acyl group, and G₂ is no residue, a hydrogen, NH₂, an aliphatic amine of one to ten carbons, or an aromatic or arylalkyl amine. Desirably, the acyl group is an acetyl, propionyl, butanyl, iso-propionyl, or iso-butanyl; the aliphatic amine of one to ten carbons is a methylamine, iso-butylamine, iso-valerylamine, cyclopentylamine, cyclohexylmethylamine, or cyclohexylamine; and the aromatic or arylalkyl amine is aniline, napthylamine, benzylamine, cinnamylamine, or phenylethylamine.

The fourth aspect of the invention features a compound comprising a sequence (e.g., an amino acid sequence) characterized by the formula L₁P₂D₃E₄V₅T₆C₇V₈, where the compound is 25 or fewer amino acids in length, where the compound antagonizes a biological activity of an interleukin 23 (IL-23) receptor, and where;

L₁ is no residue, leucine, alanine, valine, methionine, phenylalanine, tryptophan, or φ where φ is an alpha-amino acid comprising a hydrophobic side-chain; an aliphatic amine of one to ten carbons; or an aromatic or arylalkylamine;

P₂ is no residue, proline, alanine, N-methylglycine, N-isobutylglycine, aminoisobutyric acid (Aib), N-Methyl-L-alanine (MeAla), trans-4-Hydroxyproline, diethylthiazolidine carboxylic acid (Dtc), or Ω where Ω is a conformational constraint-producing amino acid;

D₃ is no residue, aspartic acid, asparagine, glutamic acid, glutamine, serine, histidine, homoserine, beta-leucine, beta-phenylalanine, alpha amino adipic acid, or P where Ψ is a 3-amino-5-phenylpentanoic acid-alpha-amino acid comprising a hydrophobic side-chain, an aromatic amine, an aliphatic amine, or a primary arylalkyl amine;

E₄ is no residue, alanine, glutamic acid, glutamine, aspartic acid, asparagine, serine, histidine, homoserine, beta-leucine, beta-phenylalanine, alpha amino adipic acid, or Ψ where Ψ is a 3-amino-5-phenylpentanoic acid-alpha-amino acid comprising a hydrophobic side-chain, an aromatic amine, an aliphatic amine or a primary arylalkyl amine;

V₅ is no residue, valine, leucine, alanine, methionine, phenylalanine, tryptophan, or φ where φ is an alpha-amino acid comprising a hydrophobic side-chain; an aliphatic amine of one to ten carbons; or an aromatic or arylalkylamine; or a heteroaromatic or heteroarylalkylamine;

T₆ is no residue, threonine, phenylalanine, alanine, or Σ where Σ defines an alpha-amino acid comprising a hydrophobic side-chain Σ or aromatic side chain;

C₇ is no residue, cysteine, serine, homoserine, homocysteine, threonine, methionine, N-acetylcysteine, cystathionine, 2-aminobutyrate, or β,β-dimethylcysteine (Penicillamine); and

V₈ is no residue, valine, leucine, alanine, methionine, phenylalanine, tryptophan, or φ where φ is an alpha-amino acid comprising a hydrophobic side-chain; an aliphatic amine of one to ten carbons; or an aromatic or arylalkylamine.

In a desirable embodiment of the fourth aspect of the invention, the compound comprises at least one D-amino acid. Desirably, the compound of the fourth aspect of the invention comprises two, three, four, five, six, seven, or even eight D-amino acids or consists entirely of D-amino acids. In another desirable embodiment, the compound is a reverse-D or reverse-L peptide or a reverse peptide containing a combination of L- and D-amino acids.

In other desirable embodiments of the fourth aspect of the invention, the alpha-amino acid comprising a hydrophobic side-chain is nor-leucine, iso-leucine, tert-leucine, cyclohexylalanine, or allylglycine; the aliphatic amine of one to ten carbons is a methylamine, iso-butylamine, iso-valerylamine, cyclopentylamine, cyclohexylmethylamine, or cyclohexylamine; and the aromatic or arylalkylamine is aniline, naphtylamine, benzylamine, cinnamylamine, or phenylethylamine.

In additional desirable embodiments of the fourth aspect of the invention, the alpha-amino acid comprising a hydrophobic side-chain Σ or aromatic side chain is nor-leucine, iso-leucine, tert-leucine, cyclohexylalanine, allylglycine, napthylalanine, pyridylalanine, histidine, tyrosine, alanine, valine, isoleucine, leucine, methionine, phenylalanine, tryptophan, or Λ where Λ is a neutral aliphatic amino acid; an aliphatic amine of one to ten carbons; an aromatic or arylalkylamine; tyrosine; 4-hydroxyphenylglycine; phenylglycine; homoserine; 3,4-dihydroxyphenylalanine; or 4-chlorophenylalanine.

In further desirable embodiments of the fourth aspect of the invention, the aliphatic amine of one to ten carbons is a methylamine, iso-butylamine, iso-valerylamine, cyclopentylamine, cyclohexylmethylamine, or cyclohexylamine; the aromatic or arylalkylamine is aniline, naphtylamine, benzylamine, cinnamylamine, or phenylethylamine; the conformational constraint-producing amino acid is azetidine-2-carboxylic acid, pipecolic acid, isonipecotic acid, 4-(aminomethyl)benzoic acid, 2-aminobenzoic acid, or nipecotic acid; and the primary arylalkyl amine is a benzylamine, phenylethylamine, 2,2-diphenylethylamine, or 4-phenyl-benzylamine.

In yet other desirable embodiments of the fourth aspect of the invention, the compound further comprises G₁ attached to the amino-terminus of the sequence, G₂ attached to the carboxy-terminus of the sequence, or G₁ attached to the amino-terminus of the sequence and G₂ attached to the carboxy-terminus of the sequence, where G₁ is no residue, a hydrogen, a straight chained or branched alkyl group of one to eight carbons, or an acyl group, and G₂ is no residue, a hydrogen, NH₂, an aliphatic amine of one to ten carbons, or an aromatic or arylalkyl amine. Desirably, the acyl group is an acetyl, propionyl, butanyl, iso-propionyl, or iso-butanyl; the aliphatic amine of one to ten carbons is a methylamine, iso-butylamine, iso-valerylamine, cyclopentylamine, cyclohexylmethylamine, or cyclohexylamine; and the aromatic or arylalkyl amine is aniline, napthylamine, benzylamine, cinnamylamine, or phenylethylamine.

The fifth aspect of the invention features a vector comprising an isolated nucleic acid sequence encoding the amino acid sequence of any one of SEQ ID NOS:1-5. The sixth aspect of the invention features a cell comprising the vector of the fifth aspect of the invention. In desirable embodiments of the sixth aspect of the invention, the cell is a prokaryotic cell or a eukaryotic cell.

The seventh aspect of the invention features a cell expressing the compound of any one of the first four aspects or the eleventh aspect of the invention. In desirable embodiments of the seventh aspect of the invention, the cell is a prokaryotic cell or a eukaryotic cell.

The eighth aspect of the invention features a pharmaceutical composition comprising the compound of any one of the first four aspects or the eleventh aspect of the invention.

The ninth aspect of the invention features a method of treating an autoimmune or inflammatory disorder. This method comprises administering to a subject in need thereof an effective dose of the compound of any one of the first four aspects of the invention. In desirable embodiments of the ninth aspect of the invention, the autoimmune or inflammatory disorder is inflammatory bowel disease, psoriasis, or multiple sclerosis. In another desirable embodiment of the ninth aspect of the invention, the compound is administered in combination with an anti-inflammatory compound. Desirably, the compound is administered orally.

The tenth aspect of the invention features a method of identifying a candidate compound that inhibits or enhances the ability of the compound of any one of the first four aspects of the invention to antagonize a biological activity of an interleukin 23 receptor. This method comprises (i) contacting the interleukin 23 receptor with the candidate compound in the presence of the compound of any one of the first four aspects of the invention; and (ii) assaying for an increase or decrease of the biological activity of the interleukin 23 receptor relative to a control not contacted with the candidate compound, where a decrease of the biological activity relative to the control indicates that the candidate compound enhances the ability of the compound of any one of the first four aspects of the invention to antagonize a biological activity of an interleukin 23 receptor, and where an increase of the biological activity relative to the control indicates that the candidate compound inhibits the ability of the compound of any one of the first four aspects of the invention to antagonize a biological activity of an interleukin 23 receptor.

In desirable embodiments of the tenth aspect of the invention, the compound of any one of the first four aspects of the invention is labeled with a moiety, which directly or indirectly provides a detectable signal. Desirably, the moiety is a radiolabel, such as ¹²⁵I, ¹⁴C, or ³H or the moiety is alkaline phosphatase or horseradish peroxidase.

The eleventh aspect of the invention features a compound comprising a sequence (e.g., an amino acid sequence) characterized by the formula D₁L₂S₃S₄G₅Y₆P₇P₈D₉I₁₀, where the compound is 25 or fewer amino acids in length, where the compound agonizes a biological activity of an interleukin 23 (IL-23) receptor, and where;

D₁ is no residue, aspartic acid, asparagine, glutamic acid, glutamine, serine, histidine, homoserine, beta-leucine, beta-phenylalanine, alpha amino adipic acid, or Ψ; where Ψ is a 3-amino-5-phenylpentanoic acid-alpha-amino acid comprising a hydrophobic side-chain, an aromatic amine, an aliphatic amine, or a primary arylalkyl amine;

L₂ is no residue, leucine, alanine, valine, methionine, phenylalanine, tryptophan, or φ where φ is an alpha-amino acid comprising a hydrophobic side-chain; an aliphatic amine of one to ten carbons; or an aromatic or arylalkylamine;

S₃ and S₄ each independently is no residue, serine, threonine, allothreonine, hydroxyproline, beta-hydroxyvaline, valine, or η, where η is a neutral hydrophilic amino acid;

G₅ is no residue, glycine, alanine, isoleucine valine, leucine, methionine, phenylalanine, tryptophan, or φ where φ is an alpha-amino acid comprising a hydrophobic side-chain; an aliphatic amine of one to ten carbons; or an aromatic or arylalkylamine;

Y₆ is no residue, tyrosine, phenylalanine, tryptophan, alanine, or Σ, where Σ is an alpha-amino acid comprising a hydrophobic side-chain Σ or aromatic side chain;

P₇ and P_{g} each independently is no residue, proline, alanine, N-methylglycine, N-isobutylglycine, aminoisobutyric acid (Aib), N-Methyl-L-alanine (MeAla), trans-4-Hydroxyproline, diethylthiazolidine carboxylic acid (Dtc), or Ω where Ω is a conformational constraint-producing amino acid;

D₉ is no residue, aspartic acid, asparagine, glutamic acid, glutamine, serine, histidine, homoserine, beta-leucine, beta-phenylalanine, alpha amino adipic acid, or Ψ; where Ψ is a 3-amino-5-phenylpentanoic acid-alpha-amino acid comprising a hydrophobic side-chain, an aromatic amine, an aliphatic amine, or a primary arylalkyl amine; and

I₁₀ is no residue, isoleucine valine, leucine, alanine, methionine, phenylalanine, tryptophan, or φ where φ is an alpha-amino acid comprising a hydrophobic side-chain; an aliphatic amine of one to ten carbons; or an aromatic or arylalkylamine.

In a desirable embodiment of the eleventh aspect of the invention, the compound includes at least one D-amino acid. In other desirable embodiments, the compound of the eleventh aspect of the invention includes two, three, four, five, six, seven, eight, nine, or even ten D-amino acids or consists entirely of D-amino acids. In another desirable embodiment, the compound is a reverse-D or reverse-L peptide or a reverse peptide containing a combination of L- and D-amino acids.

In other desirable embodiments of the eleventh aspect of the invention, the neutral amino acid is hydroxyvaline, beta,beta-dialkylserine, homo-serine, allothreonine, or hydroxyproline; the alpha-amino acid comprising a hydrophobic side-chainis nor-leucine, iso-leucine, tert-leucine, cyclohexylalanine, or allylglycine; the aliphatic amine of one to ten carbons is a methylamine, iso-butylamine, iso-valerylamine, cyclopentylamine, cyclohexylmethylamine, or cyclohexylamine; and the aromatic or arylalkylamine is aniline, naphtylamine, benzylamine, cinnamylamine, or phenylethylamine.

In additional desirable embodiments of the eleventh aspect of the invention, the alpha-amino acid comprising a hydrophobic side-chain Σ or aromatic side chain is nor-leucine, iso-leucine, tert-leucine, cyclohexylalanine, allylglycine, napthylalanine, pyridylalanine, histidine, tyrosine, alanine, valine, isoleucine, leucine, methionine, phenylalanine, tryptophan, or Λ, where Λ is a neutral aliphatic amino acid; an aliphatic amine of one to ten carbons; an aromatic or arylalkylamine; tyrosine; 4-hydroxyphenylglycine; phenylglycine; homoserine; 3,4-dihydroxyphenylalanine; or 4-chlorophenylalanine.

In yet other desirable embodiments of the eleventh aspect of the invention, the conformational constraint-producing amino acid is azetidine-2-carboxylic acid, pipecolic acid, isonipecotic acid, 4-(aminomethyl)benzoic acid, 2-aminobenzoic acid, or nipecotic acid; and the primary arylalkyl amine is a benzylamine, phenylethylamine, 2,2-diphenylethylamine, or 4-phenyl-benzylamine.

In further desirable embodiments of the eleventh aspect of the invention, the compound further comprises G₁ attached to the amino-terminus of the sequence, G₂ attached to the carboxy-terminus of the sequence, or G₁ attached to the amino-terminus of the sequence and G₂ attached to the carboxy-terminus of the sequence, where G₁ and is no residue, a hydrogen, a straight chained or branched alkyl group of one to eight carbons, or an acyl group, and G₂ is no residue, a hydrogen, NH₂, an aliphatic amine of one to ten carbons, or an aromatic or arylalkyl amine. Desirably, the acyl group is an acetyl, propionyl, butanyl, iso-propionyl, or iso-butanyl; the aliphatic amine of one to ten carbons is a methylamine, iso-butylamine, iso-valerylamine, cyclopentylamine, cyclohexylmethylamine, or cyclohexylamine, and the aromatic or arylalkyl amine is aniline, napthylamine, benzylamine, cinnamylamine, or phenylethylamine.

The twelfth aspect of the invention features a method of identifying a candidate compound that inhibits or enhances the ability of the compound of the eleventh aspect of the invention to agonize a biological activity of an interleukin 23 receptor. This method comprises (i) contacting the interleukin 23 receptor with the candidate compound in the presence of the compound of the eleventh aspect of the invention; and (ii) assaying for an increase or decrease of the biological activity of the interleukin 23 receptor relative to a control not contacted with the candidate compound, where a decrease of the biological activity relative to the control indicates that the candidate compound inhibits the ability of the compound of the eleventh aspect of the invention to agonize a biological activity of an interleukin 23 receptor, and where an increase of the biological activity relative to the control indicates that the candidate compound enhances the ability of the compound of the eleventh aspect of the invention to agonize a biological activity of an interleukin 23 receptor.

In desirable embodiments of the twelfth aspect of the invention, the compound of the eleventh aspect of the invention is labeled with a moiety, which directly or indirectly provides a detectable signal. Desirably, the moiety is a radiolabel, such as ¹²⁵I, ¹⁴C, or ³H or the moiety is alkaline phosphatase or horseradish peroxidase.

### DEFINITIONS

Unless defined otherwise, the scientific and technological terms and nomenclature used herein have the same meaning as commonly understood by a person of ordinary skill to which this invention pertains. Commonly understood definitions of molecular biology terms can be found, for example, in Singleton, et al., Dictionary of Microbiology and Molecular Biology, 2^{nd} ed. (1994, John Wiley & Sons, NY), The Harper Collins Dictionary of Biology (Hale & Marham, 1991, Harper Perennial, New York, N.Y.) Rieger et al, Glossary of genetics: Classical and molecular, 5^{th} edition, Springer-Verlag, New York, 1991; and Lewin, Genes VII, Oxford University Press, New York, 2000. Generally, the procedures of cell cultures, infection, molecular biology methods and the like are common methods used in the art. Such standard techniques can be found in reference manuals such as, for example, Ausubel et al., Current Protocols in Molecular Biology, Wiley Interscience, New York, 2001; and Sambrook et al., Molecular Cloning: A Laboratory Manual, 3^{rd} edition, Cold Spring Harbor Laboratory Press, N.Y., 2001.

As used herein, the twenty natural amino acids and their abbreviations follow conventional usage. Unconventional amino acids and their stereoisomers, e.g., D=amino acids such as α,α-disubstituted amino acids, N-alkyl amino acids, lactic acid, and others may also be suitable components for the polypeptides of the present invention. Examples of unconventional amino acids include, but are not limited to, citrulline, ornithine, norvaline, 4-(E)-butenyl-4(R)-methyl-N-methylthreonine (MeBmt), N-methyl-leucine (MeLeu), aminoisobutyric acid, statine, and N-methyl-alanine (MeAla).

The term “aromatic amines” as used herein refers to a molecule having a ring of 6 to 10 carbon atoms. Exemplary aromatic amines include, but are not limited to, phenylmethylamine, phenylethylamine, phenylpropylamine, and an amine comprising a saturated or unsaturated hydrocarbon chain.

The term “arylalkylamine” as used herein refers to an amine containing a saturated or unsaturated hydrocarbon chain. A primary arylalkylamine is composed of a ring of 6 to 10 carbon atoms. Exemplary arylalkylamines include but are not limited to phenyl, tolyl, alkoxyphenyl, alkoxycarbonylphenyl, and halophenyl.

The term “aryl” as used herein, is phenyl, 1-naphthyl, and 2-naphthyl. The term “substituted aryl” as used herein, is phenyl, 1-naphthyl and 2-naphthyl having a substituent selected from the group consisting of phenyl, heteroaryl, lower alkyl, lower alkoxy, lower alkylthio, halo, hydroxy, trifluoromethyl, amino, —NH(lower alkyl), and —N(lower alkyl)₂, as well as being mono-, di- and tri-substituted phenyl, 1-naphthyl, and 2-naphthyl containing substituents selected from methyl, methoxy, methylthio, halo, hydroxy, and amino.

The term “alkyl” as used herein, refers to straight or branched chain radicals having up to seven carbon atoms. The term “lower alkyl” as used herein, refers to straight or branched radicals having up to four carbon atoms and is a desirable sub-grouping for the term “alkyl.”

The term “substituted alkyl” as used herein, refers to straight or branched chain radicals having up to 7 carbon atoms where one or more, desirably one, two, or three hydrogen atoms have been replaced by a substituent selected from the group consisting of hydroxy, amino, cyano, halogen, trifluoromethyl, —NH(lower alkyl), —N(lower alkyl)₂, lower alkoxy, lower alkylthio, and carboxy, aryl, and heteroaryl.

As used herein, the twenty naturally-occurring L-amino acids and their abbreviations follow conventional usage. In the polypeptide notation used herein, the left-hand direction is the amino-terminal direction and the right-hand direction is the carboxy-terminal direction, in accordance with standard usage and convention.

As used herein, the terms “peptides” and “polypeptides” refer to macromolecules which comprise a multiplicity of amino or imino acids (or their equivalents) in peptide linkage. Peptides are desirably 25 or fewer amino acids in length. More desirably, peptides are between 5 and 15 or between 5 and 10 amino acids in length (e.g., 5, 6, 7, 8, 9, 10, 11, 12, 13, 14, or 15 amino acids in length). Peptides or polypeptides may include or lack posttranslational modifications. In desirable embodiments, the peptide is derived from a flexible region of an IL-23 receptor and, desirably, is chosen so that the peptide is complementary to the flexible region and follows the contours of the targeted domain. Desirably, peptides and polypeptides are IL-23 receptor subfragment peptides, such as D-amino acid antagonist peptides and other derivatives of the peptides that are capable of inhibiting IL-23 receptor activity. Desirably a peptide derivative contains a D-amino acid at the N-terminal or the C-terminal amino acid. In desirable embodiments, a peptide is the 2305, 2307, 2309, 2303, or 2301 peptide or a peptide of Formula I, II, III, IV, or V described herein. Exemplary modifications include N-terminal acetylation, glycosylation, PEGylation, and biotinylation. For example, a polypeptide may be modified to enhance stability without altering the biological activity of the interaction domain.

In addition, a peptide may be constituted of the sequences of two peptides having separately the property of inhibiting the activation (e.g., oligomerization) of a particular cytokine receptor, but not being contiguous within the flexibility regions. Such peptides can also be described as having a sequence corresponding to the particular cytokine receptor with an internal deletion.

The term “reverse-D peptide” as used herein refers to peptides containing D-amino acids, arranged in a reverse sequence relative to a peptide containing L-amino acids. For example, the C-terminal residue of an L-amino acid peptide becomes N-terminal for the D-amino acid peptide, and so forth. Reverse D-peptides desirably retain the same tertiary conformation and therefore the same activity, as the L-amino acid peptides, but desirably are more stable to enzymatic degradation in vitro and in vivo, and therefore can have greater therapeutic efficacy than the original peptide (Brady and Dodson, Nature 368:692-693, 1994; and Jameson and McDonnel, Nature 368:744-746, 1994).

The term “reverse-L peptide” as used herein refers to peptides containing L-amino acids arranged in a reverse sequence relative to a parent peptide. The C-terminal residue of the parent peptide becomes N-terminal for the reverse-L peptide, and so forth.

As used herein “antagonist,” “peptide antagonist,” or “IL-23 receptor antagonist” refers to a compound capable of inhibiting (completely or partially) a biological activity of an IL-23 receptor. The terms “antagonist,” “peptide antagonist” or “IL-23 receptor antagonist” also include potentiators of known compounds with antagonist properties.

As used herein “agonist,” “peptide agonist,” or “IL-23 receptor agonist” refers to a compound capable of stimulating a biological activity of an IL-23 receptor. The terms “agonist,” “peptide agonist” or “IL-23 receptor agonist” also include potentiators of known compounds with agonist properties.

As used herein, the designation “functional derivative” denotes, in the context of a functional derivative of an amino acid sequence, a molecule that retains a biological activity (either function or structural) that is substantially similar to that of the original sequence. Desirably, the functional derivative or equivalent is a natural derivative or is prepared synthetically. Exemplary desirable functional derivatives include amino acid sequences having substitutions, deletions, or additions of one or more amino acids, provided that the biological activity of the protein is conserved (e.g., it acts as a non-competitive antagonist of a IL-23 receptor). The substituting amino acid desirably has chemico-physical properties which are similar to that of the substituted amino acid. Desirable similar chemico-physical properties include, similarities in charge, bulkiness, hydrophobicity, hydrophylicity, and the like. The term “functional derivatives” further includes “fragments,” “analogs” or “chemical derivatives” of the IL-23 receptor binding peptides disclosed herein.

The terms “biological activity” or “IL-23 receptor activity” refers to any detectable biological activity of an IL-23 receptor. The activity desirably includes a specific biological activity of the IL-23 receptor protein, such as measurement of IL-23-induced TNFα or STAT3 (signal transducer and activator of transcription 3) phosphorylation. Biological activity also includes, for example, binding of compounds, substrates, interacting proteins and the like to IL-23 receptor. For example, measuring the effect of a test compound on its ability to inhibit or increase (i.e., modulate IL-23 response or IL-23 receptor binding or interaction), involves measuring a biological activity of IL-23 receptor according to the present invention. IL-23 receptor activity or biological activity also includes any biochemical measurement of this receptor, conformational changes, phosphorylation status, any downstream effect of the receptor signaling such as protein phosphorylation (or any other posttranslational modification e.g., ubiquitination, sumolylation, palmytotoylation, prenylation etc.), kinase effect or any other feature of the peptide that can be measured with techniques known in the art.

The biological activity of an IL-23 receptor may be measured using a variety of methods standard in the art including the phosphorylation, TNFα generation, and histological assays described herein.

The term “variant” as used herein in connection with an amino acid sequence, refers to a peptide or polypeptide that is substantially identical in structure and maintains at least one of the biological activities of the peptide or polypeptide on which it is based. Similarly, the term “variant” as used herein in connection with a nucleic acid sequence refers to a nucleic acid sequence that is substantially identical in structure to the nucleic acid sequence on which it is based and encodes a peptide or polypeptide that has at least one of the biological activities of the peptide or polypeptide encoded by the nucleic acid sequence on which the variant is based.

The term “subject” or “patient” as used herein refers to a mammal, desirably a human, who is the object of treatment, observation or experiment.

The terms “inhibiting,” “reducing,” “preventing,” or “antagonizing,” or any variations of these terms as used herein, refer to a measurable decrease of a biological activity. Desirably the decrease is a 20%, 40%, 60%, 80%, 90%, or 95% reduction in the biological activity relative to a control. Desirably, the measurable decrease is complete inhibition of the biological activity. For example, a peptide, a peptide derivative or a peptidomimetic is found to inhibit IL-23 receptor activity when a decrease in TNFα generation or STAT3 phosphorylation is measured following contacting the cell with the peptide, peptide derivative or peptidomimetic, in comparison to a control cell not contacted with the peptide, peptide derivative or peptidomimetic.

The terms “stimulating,” “increasing,” or “agonizing,” or any variations of these terms as used herein, refer to a measurable increase of a biological activity. Desirably the increase is a 20%, 40%, 60%, 80%, 90%, or 95% increase in the biological activity relative to a control. For example, a peptide, a peptide derivative or a peptidomimetic is found to stimulate IL-23 receptor activity when a increase in TNFα generation or STAT3 phosphorylation is measured following contacting the cell with the peptide, peptide derivative or peptidomimetic, in comparison to a control cell not contacted with the peptide, peptide derivative or peptidomimetic.

As used herein, the term “purified” refers to a molecule (e.g., a IL-23 receptor, a peptide, a peptide derivatives, a peptidomimetic, or a nucleic acid sequence) separated from other components that naturally accompany it. Thus, for example, a “purified peptide” has been purified to a level not found in nature. A “substantially pure” molecule is a molecule that is lacking in most other components that naturally accompany it, for example, a molecule that is 50%, 70%, 75%, 80%, 85%, 90%, 95%, 96%, 97%, 98%, 99%, or even 100% by weight, pure. A substantially pure peptide may be obtained by chemical synthesis, separation of the peptide from natural sources, or production of the peptide in a recombinant host cell that does not naturally produce the peptide.

By “isolated” in reference to a nucleic acid sequence is meant a nucleic acid sequence that is free of the nucleic acid sequences which, in the naturally-occurring gene from which the isolated nucleic acid sequence is derived, flank the nucleic acid sequence. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. It also includes a recombinant DNA that is part of a hybrid gene encoding additional polypeptide sequence.

In contrast, the term “crude” means a compound that has not been separated from the components that naturally accompanies it. Therefore, the terms “separating” or “purifying” refers to methods by which one or more components of the biological sample are removed from one or more other components of the sample. A compound, for example, a peptide, may be purified by one skilled in the art using standard techniques, such as those described by Ausubel et al. (Current Protocols in Molecular Biology, John Wiley & Sons, New York, 2000). The compound is preferably at least 2, 5, or 10-times as pure as the starting material, as measured using polyacrylamide gel electrophoresis, column chromatography, optical density, HPLC analysis, or Western analysis (Ausubel et al. Current Protocols in Molecular Biology, John Wiley & Sons, New York, 2000). Preferred methods of purification include salt precipitation, gel filtration, hydrophobic interaction chromatography, ion exchange chromatography, lectin chromatography, reversed phase chromatography, as well as combinations of these methods. Exemplary components separated from a peptide include nucleic acids in a generally aqueous solution that may include other components, such as proteins, carbohydrates, or lipids.

By “substantially identical” is meant a polypeptide or nucleic acid sequence exhibiting at least 40%, preferably 50%, 60%, 70%, 75%, or 80%, more preferably 85%, 90% or 95%, and most preferably 99% identity to a reference amino acid or nucleic acid sequence. For polypeptides, the length of comparison sequences generally is at least 5, 6, 7, 8, 9, 10, or 15 contiguous amino acids, preferably at least 20 contiguous amino acids, more preferably at least 25, 50, 75, 90, 100, 150, 200, 250, or 300 contiguous amino acids, and most preferably the full-length amino acid sequence. For nucleic acids, the length of comparison sequences generally is at least 45 contiguous nucleotides, preferably at least 60 contiguous nucleotides, more preferably at least 75, 150, 250, 300, 450, 600, 750, or 900 contiguous nucleotides, and most preferably the full-length nucleotide sequence.

The term “pharmaceutically acceptable carrier” refers to a carrier medium which does not interfere with the effectiveness of the biological activity of a peptide, peptide derivative or peptidomimetic and which is not toxic for the subject (e.g., a human patient) to whom it is administered.

A “therapeutically effective” or “pharmaceutically effective” amount refers to an amount of a peptide, peptide derivative or peptidomimetic of the present invention that is sufficient to induce a desired effect. Such result can be alleviation or reduction of the signs, symptoms or causes of a disorder or any other desired alteration of the target physiological system. For example, the compounds of the present invention have therapeutic value in the treatment of autoimmune or inflammatory disorders in which the physiology or homeostasis of the cell and/or tissue is compromised by a defect in IL-23 or IL-23 receptor production or response. Desirably the autoimmune disorder is inflammatory bowel disease, an inflammatory skin disorder such as psoriasis, or multiple sclerosis.

An “autoimmune disorder” as used herein refers to a disorder resulting from attack of a body's own tissue by its immune system. Desirably, the autoimmune disease is diabetes melitus, multiple sclerosis, premature ovarian failure, scleroderma, Sjogren's disease, lupus, alopecia (baldness), polyglandular failure, Grave's disease, hypothyroidism, polymyosititis, Celiac disease, Crohn's disease, inflammatory bowel disease, ulcerative colitis, autoimmune hepatitis, hypopituitarism, Guillain-Barre syndrome, myocardititis, Addison's disease, autoimmune skin diseases (e.g., psoriasis), uveititis, pernicious anemia, polymyalgia rheumatica, Goodpasture's syndrome, hypoparathyroidism, Hashimoto's thyoriditis, Raynaud's phenomenon, polymyaglia rheumatica, and rheumatoid arthritis.

An “inflammatory disorder” as used herein refers to any disease, disorder, or condition in which the immune system abnormally activated, or an injury which results in IL-23 receptor activation. Desirably, the disease, disorder, or condition is a disease of the upper and lower respiratory tract, for example, bronchial asthma, allergic asthma, non-allergic asthma, lymphomatous tracheobronchitis, allergic hypersensitivity or a hypersecretion condition, such as chronic bronchitis and cystic fibrosis; pulmonary fibrosis of various aetiologies (e.g., idiopathic pulmonary fibrosis), chronic obstructive pulmonary disease (COPD), sarcoidosis, allergic and non-allergic rhinitis; allergic or non-allergic urticaria; a skin-related diseases characterized by deregulated inflammation, tissue remodeling, angiogenesis, and neoplasm, a disease of the gastrointestinal tract, such as Crohn's disease, Hirschsprung's disease, diarrhea, malabsorption conditions, and inflammatory conditions; a disorder of the central and peripheral nervous system, such as depression, anxiety states, Parkinson's disease, migraine and other forms of cranial pain, strokes, emesis; a disease of the immune system, such as in the splenic and lymphatic tissues, an autoimmune disease or other immune-related diseases; a disease of the cardiovascular system, such as pulmonary edema, hypertension, atherosclerosis, pre-eclampsia, complex regional pain syndrome type 2, stroke and chronic inflammatory diseases such as arthritis, a bone-related diseases such as rheumatoid arthritis, as well as pain, chronic pain such as fibromyalgia, and other disorders in which the action of neurokinins, tachykinins or other related substances (e.g., hemokinins) are involved in the pathogenesis, pathology, and aetiology.

Additional examples of inflammatory disorders include acne vulgaris; acute respiratory distress syndrome; Addison's disease; allergic intraocular inflammatory diseases, ANCA-associated small-vessel vasculitis; ankylosing spondylitis; atopic dermatitis; autoimmune hemolytic anemia; autoimmune hepatitis; Behcet's disease; Bell's palsy; bullous pemphigoid; cerebral ischaemia; cirrhosis; Cogan's syndrome; contact dermatitis; Cushing's syndrome; dermatomyositis; diabetes mellitus; discoid lupus erythematosus; lupus nephritis; eosinophilic fasciitis; erythema nodosum; exfoliative dermatitis; focal glomerulosclerosis; focal segmental glomerulosclerosis; segmental glomerulosclerosis; giant cell arteritis; gout; gouty arthritis; graft-versus-host disease; hand eczema; Henoch-Schonlein purpura; herpes gestationis; hirsutism; idiopathic cerato-scleritis; idiopathic thrombocytopenic purpura; immune thrombocytopenic purpura inflammatory bowel or gastrointestinal disorders, inflammatory dermatoses; lichen planus; lymphomatous tracheobronchitis; macular edema; multiple sclerosis; myasthenia gravis; myositis; nonspecific fibrosing lung disease; osteoarthritis; pancreatitis; pemphigoid gestationis; pemphigus vulgaris; periodontitis; polyarteritis nodosa; polymyalgia rheumatica; pruritus scroti; pruritis/inflammation, psoriasis; psoriatic arthritis; pulmonary histoplasmosis; relapsing polychondritis; rosacea caused by sarcoidosis; rosacea caused by scleroderma; rosacea caused by Sweet's syndrome; rosacea caused by systemic lupus erythematosus; rosacea caused by urticaria; rosacea caused by zoster-associated pain; sarcoidosis; scleroderma; septic shock syndrome; shoulder tendinitis or bursitis; Sjogren's syndrome; Still's disease; Sweet's disease; systemic lupus erythematosus; systemic sclerosis; Takayasu's arteritis; temporal arteritis; toxic epidermal necrolysis; transplant-rejection and transplant-rejection-related syndromes; tuberculosis; type-1 diabetes; ulcerative colitis; uveitis; vasculitis; and Wegener's granulomatosis. Desirably the autoimmune disorder is inflammatory bowel disease, an inflammatory skin disorder such as psoriasis, or multiple sclerosis.

An “anti-inflammatory” compound as used herein refers to a compound that reduces inflammation in a subject. Desirably, an anti-inflammatory compound decreases IL-23 receptor activity. In desirable embodiments an anti-inflammatory compound is a steroid, such as a glucocorticoid. Desirably, the glucocorticoid is 11-alpha,17-alpha,21-trihydroxypregn-4-ene-3,20-dione; 11-beta,16-alpha,17,21-tetrahydroxypregn-4ene-3,20-dione; 11-beta,16-alpha,17,21-tetrahydroxypregn-1,4-diene-3,20-dione; 11-beta,17-alpha,21-trihydroxy-6-arpha-methylpregn-4-ene-3,20-dione; 11-dehydrocorticosterone; 11-deoxycortisol; 11-hydroxy-1,4-androstadiene-3, 17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-deoxyaldosterone; 21-deoxycortisone; 2-deoxyecdysone; 2-methylcortisone; 3-dehydroecdysone; 4-pregnene-17-alpha,20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclometasone; aldosterone; algestone; alphaderm; amadinone; amcinonide; anagestone; androstenedione; anecortave acetate; beclomethasone; betamethasone; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; ciclesonide; clobetasol; clobetasone; clobetasol propionate; clocortolone; clocortolone pivalate; clogestone; cloprednol; corticosterone; Cortisol; Cortisol acetate; Cortisol butyrate; Cortisol cypionate; Cortisol octanoate; Cortisol sodium phosphate; Cortisol sodium succinate; Cortisol valerate; 21-deoxycortisol; cortisone; cortisone acetate; cortivazol; cortodoxone; daturaolone; deflazacort; dehydroepiandrosterone; delmadinone; deoxycorticosterone; deprodone; descinolone; desonide; desoximetasone; dexafen; dexamethasone; dexamethasone 21-acetate; dexamethasone sodium phosphate; dichlorisone; diflorasone; diflorasone diacetate; diflucortolone; dihydroelatericin a; domoprednate; doxibetasol; ecdysone; ecdysterone; endrysone; enoxolone; flucinolone; fludrocortisone; fludrocortisone acetate; flugestone; flumethasone; flumethasone pivalate; flumoxonide; flunisolide; fluocinolone acetonide; fluocinolone; 9-fluorocortisone; fluocinonide; fluocortolone; fluorohydroxyandrostenedione; fluorometholone; fluorometholone acetate; fluoxymesterone; fluprednidene; fluprednisolone; flurandrenolide; flurandrenolone; fluticasone; fluticasone propionate; formebolone; formestane; formocortal; gestonorone; glyderinine; halcinonide; halometasone; halopredone; haloprogesterone; hydrocortiosone cypionate; hydrocortisone 21-butyrate; hydrocortisone aceponate; hydrocortisone acetate; hydrocortisone buteprate; hydrocortisone butyrate; hydrocortisone; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; hyrcanoside; inokosterone; isoflupredone; isoflupredone acetate; isoprednidene; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednisolamate; prednisolone; prednisolone 21-hemisuccinate; prednisolone acetate; prednisolone farnesylate; prednisolone hemisuccinate; prednisolone-2 1(beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone triamcinolone acetonide; triamcinolone acetonide 21-palmitate; triamcinolone diacetate; triamcinolone hexacetonide; trimegestone; turkesterone; or wortmannin.

As used herein, the terms “compound,” “molecule,” “agent,” and “ligand” refer to natural, synthetic or semi-synthetic molecules or compounds. The term “compound” therefore denotes for example chemicals, macromolecules, cell or tissue extracts (from plants or animals) and the like. Non-limiting examples of compounds include peptides, peptide derivaties, peptidomimetics, antibodies, carbohydrates, and pharmaceutical agents. The agents can be selected and screened by a variety of means including random screening, rational selection and by rational design using, for example, protein or ligand modeling methods such as computer modeling. The terms “rationally selected” or “rationally designed” are meant to define compounds, which have been chosen based on the configuration of interacting domains of the present invention. As understood by the person of ordinary skill in the art, macromolecules having non-naturally occurring modifications are also within the scope of the term “compound.” For example, peptidomimetics, well known in the pharmaceutical industry and generally referred to as peptide analogs, can be generated by modeling as described herein.

Peptides 2301, 2303, 2305, 2307, and 2309 are also referred to herein as peptides APG-2301, APG-2303, APG-2305, APG-2307, and APG-2309, respectively.

### ADVANTAGES

Prior to the present invention, efforts to use receptors as therapeutic targets have focused on and identified analogs of their natural ligand. The ligand, by definition, interacts with the ligand binding site (orthosteric site). This approach however has often exhibited limited efficacy and/or excessive undesired side effects. In contrast, the ligands described herein can alter the signalling efficacy of the receptor while maintaining the spatio-temporal signature of the physiological response.

Also, the endogenous ligand-binding pocket of closely related receptors (e.g., receptor subtypes) are generally highly conserved (e.g., kinase domains) due to strong evolutionary pressure making it difficult to obtain orthosteric ligands of high selectivity. Allosteric sites are believed to be less conserved thus offering greater potential for developing selective molecules with reduced side effects.

Given the newly appreciated diversity of the signalling partners that can be engaged by a unique receptor leading to distinct down-stream responses (Terrillon et al., EMBO Rep. 5(1):30-4 (2004)), allosteric ligands can also offer a path to selectivity by targeting specific signalling modes of the receptor. Hence, allosteric modulation of receptors brings greater selectivity, by deciphering the conformational changes and the network of interactions that take place at the supramolecular assembly levels (Changeux et al., Science 308:1424-1428 (2005); Christopolous et al., Biochem Soc Trans 32:873-877 (2004)).

In addition, the identification of high-affinity selective orthosteric ligands has been extremely difficult for a number of receptors. This has been the case for receptors activated by lipid mediators, chemokines, growth factors, and cytokines. For instance, to date there are about 50 different chemokines and 18 chemokine receptors identified. As indicated by the disparity between the numbers of identified chemokines and chemokine receptors, these ligands often show remarkable receptor binding promiscuity. Many chemokines bind several receptors, acting as agonists on some, and as antagonists on others (Loetscher et al., J Leukoc Biol 69: 881-884 (2001)), and most receptors can bind different ligands, with different outcomes (Ogilvie et al., J Immunol 172:6715-6722 (2004)). This clearly renders the development of selective orthosteric chemokine ligands difficult. In an analogous, albeit distinct setting, the extensive spatial interaction of cytokine receptors with their ligands has precluded identification of successful orthosteric inhibitors to these receptors. Taken together, the above examples underscore the advantage of allosteric inhibitors over orthosteric inhibitors of receptors.

Other features and advantages of the invention will be apparent from the following Detailed Description, the Drawings, and the Claims.

### BRIEF DESCRIPTION OF THE DRAWINGS

FIG. 1 is a modelled graphic representation of the extracellular portion of the IL-23 receptor. (The model was constructed using Accelrys Discovery Studio 1.7 software.). At the bottom of the figure, an arrow points to plasma membrane. The other arrows indicate hinge regions targeted to produce peptides. The location of the 2303, 2305, 2307, and 2309 peptides is indicated and the amino acid sequences of these peptides are included in parentheses (SEQ ID NOS:2, 1, 3, and 4).

FIG. 2 is a graph showing the results of efficacy screening of peptides designed from the IL-23 receptor. HL-60 human monocytes were pre-incubated for 30 minutes with the particular peptides (1 μM) and stimulated for 15 minutes with IL-23 (10 ng/ml). Western Blot analysis of phosphorylated-STAT3 and total STAT3 was performed on cell lysates. Immunoblot densitometry was measured using ImagePro4+ software. Peptide 2301 exhibits agonistic properties (consistent with allosteric modulation), while peptides 2303, 2307, and to a greater extent 2305 and 2309 antagonize IL-23-induced STAT3 phosphorylation.

FIGS. 3A and 3B are graphs showing inhibition of IL-23-induced STAT3 phosphorylation and TNFα generation. FIG. 3A shows dose-dependent inhibition of IL-23 (10 ng/ml)-induced STAT3 phosphorylation in PMA-activated HL-60 monocytes. Western Blot analysis was performed on cell lysates, and immunoblot densitometry was measured using ImagePro4+ software. FIG. 3B shows dose-dependent inhibition of IL-23 (10 ng/ml)-induced TNFα production in PMA-activated HL-60 monocytes. TNFα production was quantified by ELISA 24 hours after stimulation with IL-23 using the Quantikine ELISA Kit (R&D systems). Nearly identical Emax (80-85%) and IC₅₀˜2 nM was obtained for STAT3 phosphorylation and TNFα measurements using these different parameters.

FIGS. 4A and 4B are graphs showing that peptides APG-2305 (1 μM) and APG-2309 (1 μM) inhibit IL-23-induced STAT3 phosphorylation. FIG. 4A shows results obtained using freshly isolated splenocytes and FIG. 4B shows results obtained using differentiated TH17 cells.

FIG. 5 is a graph showing dose response for APG-2305 and APG-2309 in mouse splenocytes for IL-23-induced STAT3 phosphorylation in the presence of 25 ng/ml mouse IL-23.

FIG. 6 is a graph showing the efficacy of APG-2305 derivatives in the STAT3 phosphorylation assay using mouse splenocytes.

FIG. 7 is a graph showing the efficacy of APG-2309 derivatives in the STAT3 phosphorylation assay using mouse splenocytes.

FIGS. 8A and 8B are a series of graphs and Western Blots showing the specificity and selectivity of IL-23R binding peptides. FIG. 8A shows specific binding of peptide 2305 to IL-23-expressing cells. [¹²⁵I]-2305 binding was performed in phosphate buffer saline pH=7.4 on PMA-activated HL-60 cells. [¹²⁵I]-labelled and unlabelled peptide 2305 was incubated with cells for 1 hour for equilibration. Cells were then washed four times and bound radioactivity measured using a Cobra II Autogamma gamma counter (Packard). Non-activated HL-60 cells, which express little IL-23R, were used as a negative control (shown on Western Blot of IL-23 receptor). A robust increase in IL-23R expression by PMA was observed on duplicate Western blots. Peptide 2305 is easily labelled with [¹²⁵I] on its tyrosine; in absence of tyrosine (e.g., peptide 2309) [¹⁴C]-labelled amino acids can be used. FIG. 8B shows the selectivity of IL-23 antagonistic peptides. As shown in FIG. 8B, peptide 2309 does not inhibit IL-12-induced STAT4 (signal transducer and activator of transcription 4) phosphorylation. IL-12R is very homologous to IL-23R. Western Blots of phosphorylated STAT4 and total STAT4 were performed on cell lysates.

FIGS. 9A and 9B are a series of images showing the efficacy of peptide 2309 in a TNBS-induced model of inflammatory bowel disease in rat. FIG. 9A is a photographic representation of the protective effect of peptide 2309 on macroscopic characteristics of colorectal inflammation in a TNBS-induced model of IBD in Sprague-Dawley rat. A virtual abrogation of inflammation-induced edema and redness is observed rats treated with peptide 2309. FIG. 9B shows representative histology of colonic mucosa and submucosa in a normal rat, and in a TNBS-induced model of IBD in rats treated or not treated with peptide 2309. Epithelial denudation, excessive mucosal leukocyte infiltration and engorgement of blood vessels is seen in the TNBS (10 mg)-induced IBD model, whereas peptide 2309 treatment preserves crypts and surface epithelium, and markedly diminishes mucosal leukocyte infiltration and blood vessel engorgement, to the extent of being indistinguishable from normal control gut.

FIGS. 10A and 10B are a series of images and a graph showing efficacy of topical anti-IL-23R peptides in treating PMA-induced dermatitis. PMA-induced dermatitis is often utilized as a model of chronic (and scaling) dermatitis, reminiscent of psoriatic dermatitis. FIG. 10A is a series of representative photographs demonstrating efficacy of anti-IL-23R peptides (left ears) applied topically (mixed in PEG-400) to diminish ear redness secondary to PMA-induced dermatitis in CD-1 mice. FIG. 10B is a graph showing the differences in weight (mg) of ear punches with control ears untreated with PMA. Topical administration of peptides 2305 and 2307 showed marginal efficacy, while peptide 2309 was very effective in diminishing ear edema (measured by weight change).

FIGS. 11A to 11C are a series of images and graphs showing dose-response to topical administration of peptide 2309 in PMA-induced dermatitis. FIG. 11A is a photographic representation of dose-dependent effects of topical administration of peptide 2309 (mixed in PEG-400) on PMA-induced dermatitis in CD-1 mice. Concentrations of peptide 2309≧100 nM show significant efficacy. As shown in FIG. 11B, dose-dependent reduction in PMA-induced edema is also detected by increased difference in weight (mg) of punches from ears exposed topically to PMA and ears exposed to PMA+concentrations of peptide 2309≧100 nM, and, as shown in FIG. 11C, diminished capillary leak (quantified by using Evan's blue technique) in peptide 2309-treated ears.

FIG. 12 is a series of images and graphs showing the effect of intraperitoneal injection of the 2309 peptide on PMA-induced dermatitis. On day 1, 10 μl of a 0.01% solution of PMA (v/v in acetone) was applied on the external and internal surfaces of the left ear of CD-1 mice (images in left column). Twenty-four hours later, different doses of the 2309 peptide were injected intraperitoneally (0.1 mg/kg/day; 0.5 mg/kg/day; 1 mg/kg/day bid; n=4 for each group treatment). Animals were sacrificed on day 4 (6 hours after the last 2309 injection). The weight (right column, top graph) and thickness (right column, bottom graph) of all ear punches were then determined and the peptide-treated ears with PMA were compared to PMA-only ears.

FIGS. 13A and 13B are graphs showing the effect of peptides APG-2305 (FIG. 13A) and APG-2309 (FIG. 13B) on PMA-induced inflammation. Intraperitoneal administration is abreviated “i.p.” and oral administration is abreviated “p.o.”

FIG. 14 is a graph showing the efficacy of the APG-2309 peptide in a model for Experimental Autoimmune Encephalitis (EAE). The line marked with squares represents MOG (myelin oligodendrocyte glycoprotein)-injected mice that developed the disease and the lines marked with circles and triangles represent MOG-injected mice treated with peptide APG-2309 at different concentrations and different times of intraperitoneal administration.

FIG. 15 is a series of images showing that peptide APG-2309 protects against EAE-induced demyelination of the cerebellum. The animals were treated with APG-2309 three times a day (3 mg/kg/day) and the cerebellum was stained with Sudan black stain.

FIG. 16 is a series of images showing that peptide APG-2309 protects against EAE-induced demyelination of the spinal cord. The animals were treated with APG-2309 three times a day (3 mg/kg/day) and the spinal cord was stained with Sudan black stain.

### DETAILED DESCRIPTION

The IL-23 receptor antagonists and agonists described herein possess a unique mechanism and site of action for inhibiting or stimulating, respectively, IL-23 receptor activity. In particular, antagonist and agonist peptides described herein are strategically positioned on at least one extracellular flexible region including juxtamembranous regions, flexible regions between domains of the cytokine receptor, and oligomerization site, that is important for the appropriate conformation of the receptor that enables signaling. Desirably, the flexible region is required for proper oligomerization of the receptor to occur and its resulting activation.

IL-23 receptor antagonist subfragments or peptides described herein may promote or stabilize a particular conformation of a cytokine receptor that results in inhibition or activation of the receptor activity. However, the antagonists described herein do not necessarily interfere directly with the oligomerization site. Instead, the antagonists may, for example, exert their antagonistic activity by directly or indirectly preventing the oligomerization of the complementary protein chains (of homodimers as well as heterodimers receptors) of the extracellular domain of the IL-23 receptor. This process effectively prevents activation of the intracellular receptor domains responsible for function. Subsequent signal transduction events leading to overexpression of the ligand and/or cell bound receptors responsible in part for disease expression are thereby prevented.

Alternatively, IL-23 receptor subfragment peptides or derivatives may be used to promote or stabilize the active cytokine receptor structure capable of signal transduction. Such peptides are considered agonists. Examples of agonists include peptide 2301 and compounds of Formula V described herein.

Prior to the invention described herein, the only treatments targeted against IL-23 related disorders were either monoclonal antibodies against the IL-12p40 subunit and a small molecule (STA-5326) which inhibits formation of both IL-12 and IL-23. Despite some successes in human trials using these compounds (Burakoff et al., Inflamm Bowel Dis 12:558-565 (2006); Kasper et al., Curr Med Res Opin 22:1671-1678 (2006); Krueger et al, N Engl J Med 356:580-592 (2007); Mannon et al., N Engl Med 351:2069-2079 (2004)) drawbacks include (but are not limited to) their size and costs (antibodies), and the lack of selectivity by interfering with beneficial effects of IL-12. For instance, IL-12 has an established importance in innate immunity and immunocompetence. IL-23 likely is important for the activation and recruitment of inflammatory cells required for the induction of chronic inflammation (Bowman et al., Curr Opin Infect Dis 19:245-252 (2006); Holscher, Curr Opin Investig Drugs 6:489-495 (2005); Langrish et al., Immunol Rev 202:96-105 (2004)). Hence, targeting IL-23 alone, as opposed to IL-12 and IL-23 together, should reduce treatment-related adverse effects (infections, increased risk for tumorigenesis) and avoid interfering the potential benefits of IL-12 (Becker et al., J Immunol 177:2760-2764 (2006); Camoglio et al., Eur J Immunol 32:261-269 (2002); Shiraki et al., Lab Invest 84:1491-1500 (2004). Described herein are small, selective IL-23 antagonists.

To generate selective small molecule and small peptide (<10 amino acids) inhibitors of IL-23 receptors, the platform technology described in WO 2007/004060 was used. This technology enables the generation of peptide antagonists to cytokine (and growth factor) receptors based on interference of receptor-elicited activity by targeting specific extracellular domains distinct from the ligand binding site (Garrett et al, Nature 394:395-399 (1998); Ward et al., Mol Pathol 54:125-132 (2001)). The molecules generated exhibit properties of allosteric receptor modulators and are highly specific.

A small library of kinetically stable small D-peptides(≦10 amino acids) was designed against the IL-23R subunit to specifically antagonize IL-23R function. These peptides were produced and 4 of the 9 peptides were shown to inhibit IL-23-induced STAT3 phosphorylation and tumor necrosis factor (TNF) generation in (phorbol ester)-activated human monocytes, with high efficacy (˜75-95%) and potency (IC₅₀˜2-10 nM). The peptides exhibit specific binding to IL-23-containing cells, and are selective to IL-23R as they do not interfere with IL-12-induced effects. Initial experimental results revealed robust efficacy of these small peptides in reducing inflammation in a model of inflammatory bowel disease (IBD) (TNBS-induced; Becker et al., J Immunol 177:2760-2764 (2006); Camoglio et al., Eur J Immunol 32:261-269 (2002); Zhang et al., Inflamm Bowel Dis 12:382-388 (2006)). Moreover, the experimental results demonstrate efficacy in treating PMA-induced dermatitis (model of psoriatic dermatitis; Rost, Enzymol 266:525-539 (1996); Schon, J Investig Derm 112:405-410 (1999)) when peptides of the invention are administered topically or intraperitoneally; toxicity was not detected in pilot studies.

Having demonstrated a significant effect of the IL-23R antagonist peptides described herein, structure function relationship data for these antagonists and derivatives is further explored to identify the most important regions for activity. Alanine scan mutations can be performed on peptides APG-2303, -2305, -2309, and -2307. Other amino acids can be used in the place of alanine to perform the scanning experiment. In addition, the peptides can be shortened (for example, by 1, 2, 3, or 4 amino acids at either the 3′ or the 5′ end or by deleting an internal amino acid within peptide sequence), and amino acids and amino acid analogs may be substituted in the peptides. The modified compounds can then be retested for an effect on IL-23-induced biological activity. For example, the compounds may be tested to determine their effect on IL-23 (dose response)-induced TNFα and STAT3 phosphorylation in activated macrophages (e.g., HL60 cells), and IL-23-induced IL-17 production in natural killer cells (e.g., NK-92 cells).

Further, the compounds of the invention and derivatives thereof may be further characterized by determining the selectivity of optimized anti-IL-23 compounds by determining whether they affect an IL-12 regulated pathway (e.g., STAT4 phosphorylation). Radioligand binding and chemical cross-linking can also be performed in cells transfected with IL-23R and IL-12Rβ1 subunits to determine the selectivity of the compounds for IL-23R.

Exemplary IL-23 receptor antagonists of the present invention are derived from peptides 2305, 2307, and 2309, and exemplary IL-23 receptor agonists are derived from peptide 2301 as described below. Particular derivatives of peptides APG-2305 and APG-2309, and their activity, are also described below.

### Peptide 2305

A peptide 2305 (SEQ ID NO:1) derivative which antagonizes a biological activity of the IL-23 receptor, includes the sequences characterized by the formula:

T₁E₂E₃E₄Q₅Q₆Y₇L₈ Formula I

Where the peptide desirably is 25 or fewer amino acids in length, more desirably between 5 and 15 amino acids in length, and where;

T₁ is selected from no residue, threonine, phenylalanine, alanine, and Σ, where Σ defines an alpha-amino acid comprising a hydrophobic side-chain Σ or aromatic side chain;

E₂, E₃, and E₄ each independently is no residue, alanine, glutamic acid, glutamine, aspartic acid, asparagine, serine, histidine, homoserine, beta-leucine, beta-phenylalanine, alpha amino adipic acid, or Ψ where Ψ is a 3-amino-5-phenylpentanoic acid-alpha-amino acid comprising a hydrophobic side-chain, an aromatic amine, an aliphatic amine or a primary arylalkyl amine;

Q₅ and Q₆ each independently is no residue, alanine, glutamine, aspartic acid, asparagine, glutamic acid, serine, homoserine, alpha-amino adipic acid, or ψ where ψ is a 3-amino-5-phenylpentanoic acid-alpha-amino acid comprising a hydrophobic side-chain, an aromatic amine, an aliphatic amine or a primary arylalkyl amine;

Y₇ is no residue, tyrosine, phenylalanine, tryptophan, alanine, histidine, pyridylalanine, or Σ, where Σ is an alpha-amino acid comprising a hydrophobic side-chain Σ or aromatic side chain or heteroaromatic side chain; and

L₈ is no residue, valine, leucine, alanine, methionine, phenylalanine, tryptophan, or φ, where φ is an alpha-amino acid comprising a hydrophobic side-chain; an aliphatic amine of one to ten carbons; or an aromatic or arylalkylamine; or a heteroaromatic or heteroarylalkylamine.

A compound of Formula I desirably includes at least one D-amino acid. In other desirable embodiments, two, three, four, five, six, seven, or all amino acids in Formula I are D-amino acids.

In a compound of Formula I, an alpha-amino acid comprising a hydrophobic side-chain desirably is nor-leucine, tert-leucine, cyclohexylalanine, or allylglycine. An aliphatic amine of one to ten carbons desirably is a methylamine, iso-butylamine, iso-valerylamine, cyclopentylamine, cyclohexylmethylamine, or cyclohexylamine. An aromatic or arylalkylamine desirably is aniline, naphtylamine, benzylamine, cinnamylamine, or phenylethylamine. A heteroaromatic or heteroarylalkylamine desirably is pyridylamine, pyridylmethylamine, or tryptamine. An alpha-amino acid comprising a hydrophobic side-chain Σ or aromatic side chain desirably is nor-leucine, iso-leucine, tert-leucine, cyclohexylalanine, allylglycine, napthylalanine, pyridylalanine, histidine, tyrosine, alanine, valine, isoleucine, leucine, methionine, phenylalanine, tryptophan, or Λ, where Λ is a neutral aliphatic amino acid; an aliphatic amine of one to ten carbons; an aromatic or arylalkylamine; tyrosine; 4-hydroxyphenylglycine; phenylglycine; homoserine; 3,4-dihydroxyphenylalanine; or 4-chlorophenylalanine. A primary arylalkyl amine desirably is a benzylamine, phenylethylamine, 2,2-diphenylethylamine, or 4-phenyl-benzylamine.

In a desirable embodiment, the compound of Formula I further comprises G₁ attached to the amino-terminus of the amino acid sequence, G₂ attached to the carboxy-terminus of the amino acid sequence, or G₁ attached to the amino-terminus of the amino acid sequence and G₂ attached to the carboxy-terminus of the amino acid sequence, where G₁ desirably is no residue, a hydrogen, a straight chained or branched alkyl group of one to eight carbons, or an acyl group, and G₂ desirably is no residue, a hydrogen, NH₂, an aliphatic amine of one to ten carbons, or an aromatic or arylalkyl amine or a heteroaromatic or heteroarylalkylamine. An acyl group desirably is an acetyl, propionyl, butanyl, iso-propionyl, or iso-butanyl. An aliphatic amine of one to ten carbons desirably is a methylamine, iso-butylamine, iso-valerylamine, cyclopentylamine, cyclohexylmethylamine, or cyclohexylamine. An aromatic or arylalkyl amine desirably is aniline, napthylamine, benzylamine, cinnamylamine, or phenylethylamine. A heteroaromatic or heteroarylalkylamine desirably is pyridylamine, pyridylmethylamine, or tryptamine.

In particular, to further characterize regions in peptide 2305 that are important for the modulation of its biological activity, we made mutants and truncations of this parent peptide. The derivatives of peptide 2305 are listed in Table 1 below. The biological activity of these peptides is further described in Examples 2 and 3.

TABLE 1
Exemplary derivatives of peptide 2305

| Peptide | Sequence | |
|---|---|---|
| APG-2305-1 | TEEAQQYL | SEQ ID NO: 6 |
| APG-2305-2 | TAAEQQYL | SEQ ID NO: 7 |
| APG-2305-3 | TAAAQQYL | SEQ ID NO: 8 |
| APG-2305-4 | EEEQQYL | SEQ ID NO: 9 |
| APG-2305-5 | EEQQYL | SEQ ID NO: 10 |
| APG-2305-6 | EQQYL | SEQ ID NO: 11 |
| APG-2305-7 | AEEQQYL | SEQ ID NO: 12 |
| APG-2305-8 | TEEEQQY | SEQ ID NO: 13 |
| APG-2305-9 | TEEEQQ | SEQ ID NO: 14 |
| APG-2305-10 | TEEEQ | SEQ ID NO: 15 |
| APG-2305-11 | TEEE | SEQ ID NO: 16 |
| APG-2305-12 | TEEEQAYL | SEQ ID NO: 17 |
| APG-2305-13 | TEEEAAYL | SEQ ID NO: 18 |

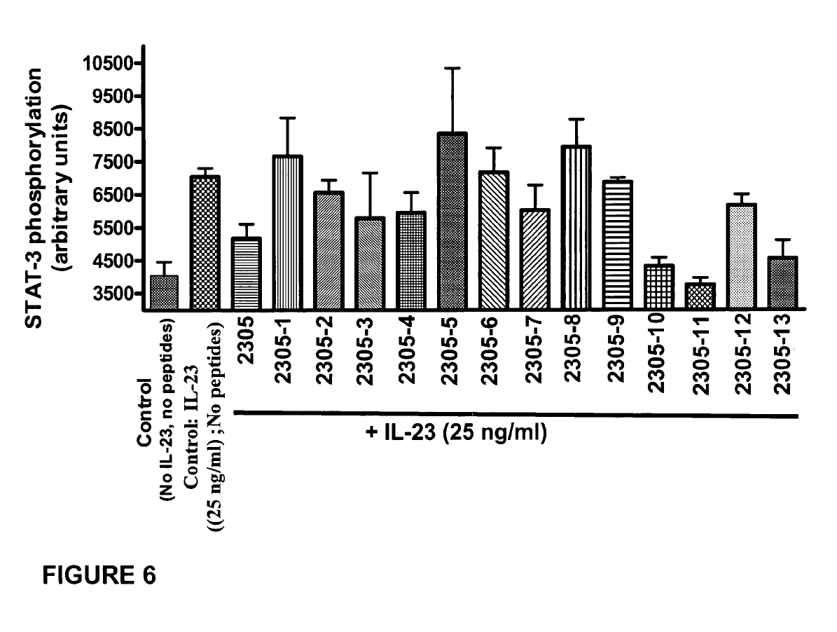

The truncation of threonine at the N-terminus as well as the partial or total removal of the negative charges of one to three glutamates in peptides 2305-1 to 2305-7 resulted in a decrease in inhibitory activity compared to the parent peptide 2305. In the case of 2305-1, the modifications result in a total loss of activity. Derivatives 2305-5 and 2305-6 showed a measurable loss of activity due to the removal of both negative charges and the N-terminal threonine. The N-terminal portion of 2305 is therefore likely important for IL-23R inhibitory activity (see FIG. 6).

While the C-terminal portion of 2305 alone does not appear to mediate the biological activity of the 2305 peptide (see FIG. 6; peptides 2305-5 and 2305-6), removal of the C-terminal tyrosine and/or the leucine abolished the activity of the 2305 peptide (see FIG. 6; peptides 2305-8 and 2305-9).

In contrast, mutations of both glutamine residues into alanine residues in the 2305 peptide increased the activity of the peptide by 25% relative to the parent 2305 peptide (see FIG. 6; peptide 2305-13). Also, a major truncation of the 2305 peptide leaving only the N-terminal portion (peptides 2305-10 and 2305-11) increased the activity. These results support an inhibitory role of the two glutamines on the activity of the 2305 peptide.

The structural characterization of the 2305 peptide indicates that the negative charges at the N-terminal of the peptide are important for the inhibition of IL-23R. In contrast, substitution of the glutamines at the C-terminal portion of the 2305 peptide increased the IL-23R inhibitory activity of the peptide relative to the parent 2305 peptide.

### Peptide 2307

A peptide 2307 (SEQ ID NO:3) derivative which antagonizes a biological activity of the IL-23 receptor, includes the sequences characterized by the formula:

K₁K₂Y₃L₄V₅W₆V₇Q₈ Formula II

Where the peptide desirably is 25 or fewer amino acids in length, more desirably between 5 and 15 amino acids in length, and where;

K₁ and K₂ each independently is no residue, lysine, arginine, histidine, alanine, ornithine, citruline, 2-pyridylalanine, 3-pyridylalanine, 4-pyridylalanine, or an arginine surrogate;

Y₃ is no residue, tyrosine, phenylalanine, tryptophan, alanine, or Σ, where Σ is an alpha-amino acid comprising a hydrophobic side-chain Σ or aromatic side chain, or heteroaromatic side chain;

L₄ is no residue, valine, leucine, alanine, methionine, phenylalanine, tryptophan, or φ, where φ is an alpha-amino acid comprising a hydrophobic side-chain; an aliphatic amine of one to ten carbons; or an aromatic or arylalkylamine; or a heteroaromatic or heteroarylalkylamine;

V₅ is no residue, valine, leucine, alanine, methionine, phenylalanine, tryptophan, or φ, where φ is an alpha-amino acid comprising a hydrophobic side-chain; an aliphatic amine of one to ten carbons; or an aromatic or arylalkylamine; or a heteroaromatic or heteroarylalkylamine;

W₆ is no residue, tryptophan, tyrosine, phenylalanine, alanine, or Σ, where Σ is an alpha-amino acid comprising a hydrophobic side-chain Σ or an aromatic side chain; or heteroaromatic side chain;

V₇ is no residue, valine, leucine, alanine, methionine, phenylalanine, tryptophan, or φ, where φ is an alpha-amino acid comprising a hydrophobic side-chain; an aliphatic amine of one to ten carbons; or an aromatic or arylalkylamine; or a heteroaromatic or heteroarylalkylamine; and

Q₈ is no residue, glutamine, aspartic acid, asparagine, glutamic acid, serine, or ψ where ψ is a 3-amino-5-phenylpentanoic acid-alpha-amino acid comprising a hydrophobic side-chain, an aromatic amine, an aliphatic amine or a primary arylalkyl amine.

A compound of Formula II desirably includes at least one D-amino acid. In other desirable embodiments, two, three, four, five, six, seven, or all amino acids in Formula II are D-amino acids.

In a compound of Formula II, an alpha-amino acid comprising a hydrophobic side-chain desirably is nor-leucine, iso-leucine, tert-leucine, cyclohexylalanine, or allylglycine. An aliphatic amine of one to ten carbons desirably is a methylamine, iso-butylamine, iso-valerylamine, cyclopentylamine, cyclohexylmethylamine, or cyclohexylamine. An aromatic or arylalkylamine desirably is aniline, naphtylamine, benzylamine, cinnamylamine, or phenylethylamine. An alpha-amino acid comprising a hydrophobic side-chain Σ or aromatic side chain desirably is nor-leucine, iso-leucine, tert-leucine, cyclohexylalanine, allylglycine, napthylalanine, pyridylalanine, histidine, tyrosine, alanine, valine, isoleucine, leucine, methionine, phenylalanine, tryptophan, or Λ, where Λ is a neutral aliphatic amino acid; an aliphatic amine of one to ten carbons; an aromatic or arylalkylamine; tyrosine; 4-hydroxyphenylglycine; phenylglycine; homoserine; 3,4-dihydroxyphenylalanine; or 4-chlorophenylalanine. An arginine surrogate desirably is 4-amidinophenylacetyl, 4-amidinophenylpropionyl, 4-amidinophenylglycyl, 4-amidinophenylmethylglycyl, 4-guanidinophenylacetyl, 4-uanidinophenylpropionyl, 4-guanidinophenylglycyl, or 4-guanidinophenylmethylglycyl (Feng and Lubell, J. Org. Chem. 66(4):1181-1185 (2001)). A primary arylalkyl amine desirably is a benzylamine, phenylethylamine, 2,2-diphenylethylamine, or 4-phenyl-benzylamine.

In a desirable embodiment, the compound of Formula II further comprises G₁ attached to the amino-terminus of the amino acid sequence, G₂ attached to the carboxy-terminus of the amino acid sequence, or G₁ attached to the amino-terminus of the amino acid sequence and O₂ attached to the carboxy-terminus of the amino acid sequence, where G₁ desirably is no residue, a hydrogen, a straight chained or branched alkyl group of one to eight carbons, or an acyl group, and G₂ desirably is no residue, a hydrogen, NH₂, an aliphatic amine of one to ten carbons, or an aromatic or arylalkyl amine. An acyl group desirably is an acetyl, propionyl, butanyl, iso-propionyl, or iso-butanyl. An aliphatic amine of one to ten carbons desirably is a methylamine, iso-butylamine, iso-valerylamine, cyclopentylamine, cyclohexylmethylamine, or cyclohexylamine. An aromatic or arylalkyl amine desirably is aniline, napthylamine, benzylamine, cinnamylamine, or phenylethylamine.

### Peptide 2309

A peptide 2309 (SEQ ID NO:4) derivative which antagonizes a biological activity of the IL-23 receptor, includes the sequences characterized by the formula:

M₁E₂E₃S₄K₅Q₆L₇Q₈L₉ Formula III

Where the peptide desirably is 25 or fewer amino acids in length, more desirably between 5 and 15 amino acids in length, and where;

M₁ is no residue, methionine, valine, leucine, alanine, phenylalanine, tryptophan, or φ where φ is an alpha-amino acid comprising a hydrophobic side-chain; an aliphatic amine of one to ten carbons; or an aromatic or arylalkylamine;

E₂ and E₃ each independently is no residue, alanine, glutamic acid, glutamine, aspartic acid, asparagine, serine, histidine, homoserine, beta-leucine, beta-phenylalanine, alpha amino adipic acid, trimesic acid, or an alpha,omega-dicarboxylic acid (e.g., succinic acid, glutaric acid, or azelic acid), or Ψ where Ψ is a 3-amino-5-phenylpentanoic acid-alpha-amino acid comprising a hydrophobic side-chain, an aromatic amine, an aliphatic amine or a primary arylalkyl amine;

S₄ is no residue, serine, threonine, allothreonine, hydroxyproline, beta-hydroxyvaline, valine, or η where η is a neutral hydrophilic amino acid;

K₅ is no residue, glutamine, lysine, arginine, histidine, alanine, ornithine, citruline, 2-pyridylalanine, 3-pyridylalanine, 4-pyridylalanine, or an arginine surrogate;

Q₆ is no residue, alanine, glutamine, aspartic acid, asparagine, glutamic acid, serine, or ψ where ψ is a 3-amino-5-phenylpentanoic acid-alpha-amino acid comprising a hydrophobic side-chain, an aromatic amine, an aliphatic amine or a primary arylalkyl amine;

L₇ is no residue, valine, leucine, alanine, methionine, phenylalanine, tryptophan, or φ where φ is an alpha-amino acid comprising a hydrophobic side-chain; an aliphatic amine of one to ten carbons; or an aromatic or arylalkylamine; or a heteroaromatic or heteroarylalkylamine;

Q₈ is no residue, glutamine, aspartic acid, asparagine, glutamic acid, serine, or ψ where ψ is a 3-amino-5-phenylpentanoic acid-alpha-amino acid comprising a hydrophobic side-chain, an aromatic amine, an aliphatic amine or a primary arylalkyl amine; and

L₉ is no residue, valine, leucine, isoleucine, alanine, methionine, phenylalanine, tryptophan, or φ where φ is an alpha-amino acid comprising a hydrophobic side-chain; an aliphatic amine of one to ten carbons; or an aromatic or arylalkylamine; or a heteroaromatic or heteroarylalkylamine.

A compound of Formula III desirably includes at least one D-amino acid. In other desirable embodiments, two, three, four, five, six, seven, eight, or all amino acids in Formula III are D-amino acids.

In a compound of Formula III, a neutral amino acid desirably is hydroxyvaline, beta,beta-dialkylserine and (as described in Dettwiler and Lubell J Org. Chem. 2003 Jan. 10; 68(1):177-9) homo-serine, allothreonine, or hydroxyproline. An alpha-amino acid comprising a hydrophobic side-chain desirably is nor-leucine, iso-leucine, tert-leucine, cyclohexylalanine, or allylglycine. An aliphatic amine of one to ten carbons desirably is a methylamine, iso-butylamine, iso-valerylamine, cyclopentylamine, cyclohexylmethylamine, or cyclohexylamine. An aromatic or arylalkylamine desirably is aniline, naphtylamine, benzylamine, cinnamylamine, or phenylethylamine. An arginine surrogate desirably is 4-amidinophenylacetyl, 4-amidinophenylpropionyl, 4-amidinophenylglycyl, 4-amidinophenylmethylglycyl, 4-guanidinophenylacetyl, 4-uanidinophenylpropionyl, 4-guanidinophenylglycyl, or 4-guanidinophenylmethylglycyl. A primary arylalkyl amine is a benzylamine, phenylethylamine, 2,2-diphenylethylamine, or 4-phenyl-benzylamine.

In a desirable embodiment, the compound of Formula III further comprises G₁ attached to the amino-terminus of the amino acid sequence, G₂ attached to the carboxy-terminus of the amino acid sequence, or G₁ attached to the amino-terminus of the amino acid sequence and G₂ attached to the carboxy-terminus of the amino acid sequence, where G₁ desirably is no residue, a hydrogen, a straight chained or branched alkyl group of one to eight carbons, or an acyl group, and G₂ desirably is no residue, a hydrogen, NH₂, an aliphatic amine of one to ten carbons, or an aromatic or arylalkyl amine. An acyl group desirably is an acetyl, propionyl, butanyl, iso-propionyl, or iso-butanyl. An aliphatic amine of one to ten carbons desirably is a methylamine, iso-butylamine, iso-valerylamine, cyclopentylamine, cyclohexylmethylamine, or cyclohexylamine. An aromatic or arylalkyl amine desirably is aniline, napthylamine, benzylamine, cinnamylamine, or phenylethylamine.

To further characterize regions in peptide 2309 that are important for the modulation of its biological activity, we made mutants and truncations of this parent peptide. The derivatives of peptide 2309 are listed in Table 2 below. The biological activity of these peptides is further described in Examples 2 and 3.

TABLE 2
Exemplary derivatives of peptide 2309

| Peptide | Sequence | |
|---|---|---|
| APG-2309-1 | MAESKQLQL | SEQ ID NO: 19 |
| APG-2309-2 | MAASKQLQL | SEQ ID NO: 20 |
| APG-2309-3 | ESKQLQL | SEQ ID NO: 21 |
| APG-2309-4 | MEESKQLQI | SEQ ID NO: 22 |
| APG-2309-5 | MEESKQL | SEQ ID NO: 23 |
| APG-2309-6 | MEESKQ | SEQ ID NO: 24 |
| APG-2309-7 | MEESQQLQI | SEQ ID NO: 25 |
| APG-2309-8 | EESKQLQL | SEQ ID NO: 26 |
| APG-2309-1-1 | VQAANALGMEESKQLQLHLDDLVL | SEQ ID NO: 27 |
| (recto) | | |
| APG-2309-2-1 | LVLDDLHLQLQKSEEMGLANAAQV | SEQ ID NO: 28 |
| (verso) | | |

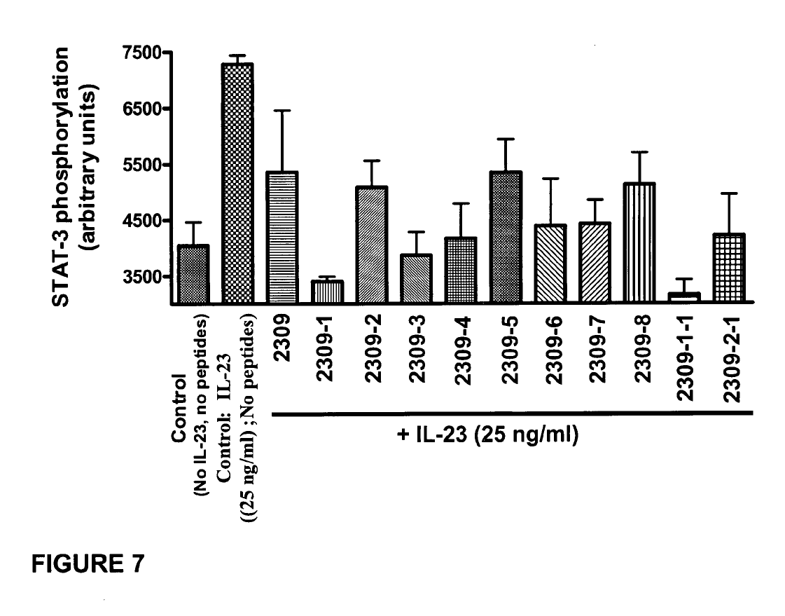

As shown in FIG. 7, the N-terminal region of the 2309 peptide is important for its IL-23R inhibitory activity. Removal or mutation of the first glutamate increases the activity (see FIG. 7; peptides 2309-1 and 2309-3). In particular, the mutation of the second glutamate into an alanine residue completely abolished the IL-23-induced activity (FIG. 7).

The 25 amino acid extended region of peptide 2309 covering part of the loop in D3 domain of IL-23R (peptide 2309-1-1) showed a 100% inhibition of STAT3 phosphorylation (50% more than the parent 2309 peptide). Interactions in this region of the IL-23R are therefore very important for the activity of IL-23R. The inverse 25 amino acid sequence (peptide 2309-2-1; a reverse-D peptide) also showed better efficacy than the parent 2309 peptide at inhibiting IL-23-induced effects. These results indicate that a longer amino acid sequence (e.g., a 25 amino acid sequence) can adopt a specific conformation that may improve the affinity and selectivity of binding to the receptor.

### Peptide 2303

A peptide 2303 (SEQ ID NO:2) derivative which antagonizes a biological activity of the IL-23 receptor, includes sequences characterized by the formula:

L₁P₂D₃E₄V₅T₆C₇V₈ Formula IV

Where the peptide desirably is 25 or fewer amino acids in length, more desirably between 5 and 15 amino acids in length, and where;

L₁ is no residue, leucine, alanine, valine, methionine, phenylalanine, tryptophan, or φ where φ is an alpha-amino acid comprising a hydrophobic side-chain; an aliphatic amine of one to ten carbons; or an aromatic or arylalkylamine;

P₂ is no residue, proline, alanine, N-methylglycine, N-isobutylglycine, aminoisobutyric acid (Aib), N-Methyl-L-alanine (MeAla), trans-4-Hydroxyproline, diethylthiazolidine carboxylic acid (Dtc), or Ω where Ω is a conformational constraint-producing amino acid;

D₃ is no residue, aspartic acid, asparagine, glutamic acid, glutamine, serine, histidine, homoserine, beta-leucine, beta-phenylalanine, alpha amino adipic acid, or Ψ where Ψ is a 3-amino-5-phenylpentanoic acid-alpha-amino acid comprising a hydrophobic side-chain, an aromatic amine, an aliphatic amine, or a primary arylalkyl amine;

E₄ is no residue, alanine, glutamic acid, glutamine, aspartic acid, asparagine, serine, histidine, homoserine, beta-leucine, beta-phenylalanine, alpha amino adipic acid, or Ψ where Ψ is a 3-amino-5-phenylpentanoic acid-alpha-amino acid comprising a hydrophobic side-chain, an aromatic amine, an aliphatic amine or a primary arylalkyl amine;

V₅ is no residue, valine, leucine, alanine, methionine, phenylalanine, tryptophan, or φ where φ is an alpha-amino acid comprising a hydrophobic side-chain; an aliphatic amine of one to ten carbons; or an aromatic or arylalkylamine; or a heteroaromatic or heteroarylalkylamine;

T₆ is no residue, threonine, phenylalanine, alanine, or Σ, where Σ defines an alpha-amino acid comprising a hydrophobic side-chain Σ or aromatic side chain;

C₇ is no residue, cysteine, serine, homoserine, homocysteine, threonine, methionine, N-acetylcysteine, cystathionine, 2-aminobutyrate, or β,β-dimethylcysteine (Penicillamine); and

V₈ is no residue, valine, leucine, alanine, methionine, phenylalanine, tryptophan, or φ where φ is an alpha-amino acid comprising a hydrophobic side-chain; an aliphatic amine of one to ten carbons; or an aromatic or arylalkylamine.

A compound of Formula IV desirably includes at least one D-amino acid. In other desirable embodiments, two, three, four, five, six, seven, or all amino acids in Formula IV are D-amino acids.

In a compound of Formula IV, an alpha-amino acid comprising a hydrophobic side-chain desirably is nor-leucine, iso-leucine, tert-leucine, cyclohexylalanine, or allylglycine. An aliphatic amine of one to ten carbons desirably is a methylamine, iso-butylamine, iso-valerylamine, cyclopentylamine, cyclohexylmethylamine, or cyclohexylamine. An aromatic or arylalkylamine desirably is aniline, naphtylamine, benzylamine, cinnamylamine, or phenylethylamine. An alpha-amino acid comprising a hydrophobic side-chain Σ or aromatic side chain desirably is nor-leucine, iso-leucine, tert-leucine, cyclohexylalanine, allylglycine, napthylalanine, pyridylalanine, histidine, tyrosine, alanine, valine, isoleucine, leucine, methionine, phenylalanine, tryptophan, or Λ where Λ is a neutral aliphatic amino acid; an aliphatic amine of one to ten carbons; an aromatic or arylalkylamine; tyrosine; 4-hydroxyphenylglycine; phenylglycine; homoserine; 3,4-dihydroxyphenylalanine; or 4-chlorophenylalanine. A conformational constraint-producing amino acid desirably is azetidine-2-carboxylic acid, pipecolic acid, isonipecotic acid, 4-(aminomethyl)benzoic acid, 2-aminobenzoic acid, or nipecotic acid (Hanessian and McNaughton-Smith, Tetrahedron 53:12789-12854, 1997; Halab et al., Biopolymers Peptide Science 55:101-122, 2000; Cluzeau and Lubell, J. Org. Chem. 69:1504-1512, 2004; Feng and Lubell, J. Org. Chem. 66:1181-1185, 2001). A primary arylalkyl amine desirably is a benzylamine, phenylethylamine, 2,2-diphenylethylamine, or 4-phenyl-benzylamine.

In a desirable embodiment, the compound of Formula IV further comprises G₁ attached to the amino-terminus of the amino acid sequence, G₂ attached to the carboxy-terminus of the amino acid sequence, or G₁ attached to the amino-terminus of the amino acid sequence and G₂ attached to the carboxy-terminus of the amino acid sequence, where G₁ desirably is no residue, a hydrogen, a straight chained or branched alkyl group of one to eight carbons, or an acyl group, and G₂ desirably is no residue, a hydrogen, NH₂, an aliphatic amine of one to ten carbons, or an aromatic or arylalkyl amine. An acyl group desirably is an acetyl, propionyl, butanyl, iso-propionyl, or iso-butanyl. An aliphatic amine of one to ten carbons desirably is a methylamine, iso-butylamine, iso-valerylamine, cyclopentylamine, cyclohexylmethylamine, or cyclohexylamine. An aromatic or arylalkyl amine desirably is aniline, napthylamine, benzylamine, cinnamylamine, or phenylethylamine.

### Peptide 2301

A peptide 2301 (SEQ ID NO:5) derivative which is an agonist of a biological activity of the IL-23 receptor, includes the sequences characterized by the formula:

D₁L₂S₃S₄G₅Y₆P₇P₈D₉I₁₀ Formula V

Where the peptide desirably is 25 or fewer amino acids in length, more desirably between 5 and 15 amino acids in length, and where;

D₁ is no residue, aspartic acid, asparagine, glutamic acid, glutamine, serine, histidine, homoserine, beta-leucine, beta-phenylalanine, alpha amino adipic acid, or Ψ; where Ψ is a 3-amino-5-phenylpentanoic acid-alpha-amino acid comprising a hydrophobic side-chain, an aromatic amine, an aliphatic amine, or a primary arylalkyl amine;

L₂ is no residue, leucine, alanine, valine, methionine, phenylalanine, tryptophan, or φ where φ is an alpha-amino acid comprising a hydrophobic side-chain; an aliphatic amine of one to ten carbons; or an aromatic or arylalkylamine;

S₃ and S₄ each independently is no residue, serine, threonine, allothreonine, hydroxyproline, beta-hydroxyvaline, valine, or η, where η is a neutral hydrophilic amino acid;

G₅ is no residue, glycine, alanine, isoleucine valine, leucine, methionine, phenylalanine, tryptophan, or φ where φ is an alpha-amino acid comprising a hydrophobic side-chain; an aliphatic amine of one to ten carbons; or an aromatic or arylalkylamine;

Y₆ is no residue, tyrosine, phenylalanine, tryptophan, alanine, or Σ, where Σ is an alpha-amino acid comprising a hydrophobic side-chain Σ or aromatic side chain;

P₇ and P₈ each independently is no residue, proline, alanine, N-methylglycine, N-isobutylglycine, aminoisobutyric acid (Aib), N-Methyl-L-alanine (MeAla), trans-4-Hydroxyproline, diethylthiazolidine carboxylic acid (Dtc), or Ω where Ω is a conformational constraint-producing amino acid;

D₉ is no residue, aspartic acid, asparagine, glutamic acid, glutamine, serine, histidine, homoserine, beta-leucine, beta-phenylalanine, alpha amino adipic acid, or Ψ where Ψ is a 3-amino-5-phenylpentanoic acid-alpha-amino acid comprising a hydrophobic side-chain, an aromatic amine, an aliphatic amine, or a primary arylalkyl amine; and

I₁₀ is no residue, isoleucine valine, leucine, alanine, methionine, phenylalanine, tryptophan, or φ where φ is an alpha-amino acid comprising a hydrophobic side-chain; an aliphatic amine of one to ten carbons; or an aromatic or arylalkylamine.

A compound of Formula V desirably includes at least one D-amino acid. In other desirable embodiments, two, three, four, five, six, seven, eight, nine, or all amino acids in Formula V are D-amino acids.

In a compound of Formula V, a neutral amino acid desirably is hydroxyvaline, beta,beta-dialkylserine, homo-serine, allothreonine, or hydroxyproline. An alpha-amino acid comprising a hydrophobic side-chain desirably is nor-leucine, iso-leucine, tert-leucine, cyclohexylalanine, or allylglycine. An aliphatic amine of one to ten carbons desirably is a methylamine, iso-butylamine, iso-valerylamine, cyclopentylamine, cyclohexylmethylamine, or cyclohexylamine. An aromatic or arylalkylamine desirably is aniline, naphtylamine, benzylamine, cinnamylamine, or phenylethylamine. An alpha-amino acid comprising a hydrophobic side-chain Σ or aromatic side chain desirably is nor-leucine, iso-leucine, tert-leucine, cyclohexylalanine, allylglycine, napthylalanine, pyridylalanine, histidine, tyrosine, alanine, valine, isoleucine, leucine, methionine, phenylalanine, tryptophan, or Λ where Λ is a neutral aliphatic amino acid; an aliphatic amine of one to ten carbons; an aromatic or arylalkylamine; tyrosine; 4-hydroxyphenylglycine; phenylglycine; homoserine; 3,4-dihydroxyphenylalanine; or 4-chlorophenylalanine. A conformational constraint-producing amino acid desirably is azetidine-2-carboxylic acid, pipecolic acid, isonipecotic acid, 4-(aminomethyl)benzoic acid, 2-aminobenzoic acid, or nipecotic acid. A primary arylalkyl amine desirably is a benzylamine, phenylethylamine, 2,2-diphenylethylamine, or 4-phenyl-benzylamine.

In a desirably embodiment, the compound of Formula V further comprises G₁ attached to the amino-terminus of the amino acid sequence, G₂ attached to the carboxy-terminus of the amino acid sequence, or G₁ attached to the amino-terminus of the amino acid sequence and G₂ attached to the carboxy-terminus of the amino acid sequence, where G₁ and is no residue, a hydrogen, a straight chained or branched alkyl group of one to eight carbons, or an acyl group, and G₂ is no residue, a hydrogen, NH₂, an aliphatic amine of one to ten carbons, or an aromatic or arylalkyl amine. An acyl group desirably is an acetyl, propionyl, butanyl, iso-propionyl, or iso-butanyl. An aliphatic amine of one to ten carbons desirably is a methylamine, iso-butylamine, iso-valerylamine, cyclopentylamine, cyclohexylmethylamine, or cyclohexylamine. An aromatic or arylalkyl amine desirably is aniline, napthylamine, benzylamine, cinnamylamine, or phenylethylamine.

### Assays to Identify Antagonist or Agonist Peptides

Screens for IL-23 receptor antagonists or agonists (e.g., candidate or test compounds or agents like peptides, peptidomimetics, small molecule or other drugs) may be based on assays which measure a biological activity of an IL-23 receptor. The assays described herein desirably employ a natural or a recombinant IL-23 receptor. A cell fraction or cell free screening assay for antagonists or agonists of cytokine activity can use in situ purified, or purified recombinant cytokine receptor. Cell-based assays can employ cells which express the cytokine receptor naturally, or which contain the recombinant cytokine receptor. In all cases, the biological activity of the cytokine receptor can be directly or indirectly measured. Thus inhibitors or activators of cytokine receptor activity can be identified. The inhibitors or activators themselves may be further modified by standard combinatorial chemistry techniques to provide improved analogs of the originally identified compounds.

The compounds of the present invention are useful in vitro as unique tools for understanding the biological role of IL-23 as well as the many factors thought to influence and be influenced by the production of IL-23 and its binding to its receptor. The antagonists and agonists of the present invention are also useful in the development of other compounds that bind the IL-23 receptor because the peptide antagonists (e.g., peptides 2303, 2305, 2307, and 2309) and agonists (e.g., peptide 2301) of the present invention provide important information on the relationship between structure and activity that can facilitate such development.

For example, the compounds described herein can be used as competitive inhibitors in assays to screen for, or to characterize similar new peptide receptors antagonists or agonists. In such assays, as well as assays for determining IL-23 receptor expression, the peptides or peptidomimetics of the present invention can be used without modification or they can be labeled (i.e., covalently or non-convalently linked to a moiety which directly or indirectly provide a detectable signal). Examples of labels include radiolabels such as ¹²⁵I, ¹⁴C, and ³H, enzymes such as alkaline phosphatase and horseradish peroxidase (U.S. Pat. No. 3,645,090), ligands such as biotin and avidin, and luminescent compounds including bioluminescent, phosphorescent, chemiluminescent or fluorescent labels (U.S. Pat. No. 3,940,475).

Alternatively, determining the ability of the test compound to modulate the activity of the IL-23 receptor complex can be accomplished by determining the ability of the test compound to modulate the activity of a downstream effector of an IL-23 receptor target molecule. For instance, the activity of the test compound on the effector molecule may be determined.

Those skilled in the field of drug discovery and development understand that the precise source of test compounds is not critical to the methods of the invention. Examples of such test compounds include, but are not limited to, plant-, fungal-, prokaryotic-, or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceanographics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

For example, to identify compounds that modulate the activity of an IL-23 receptor, or that inhibit or enhance the ability of a compound described herein to inhibit or stimulate an IL-23 receptor biological activity, a cell-based assay may be used in which a cell expressing the IL-23 receptor complex or biologically active portion thereof (either natural or recombinant) is contacted with a test compound to determine the ability of the test compound to modulate the cytokine receptor biological activity. The cell-based assays include proliferation assays, tyrosine phosphorylation assays, migration assays, and any other assay that measures a biological activity of the cytokine receptor.

In assays for measuring the activity of a test compound, it is desirable to immobilize the IL-23 receptor, or an interacting peptide or peptidomimetic of the present invention, to facilitate separation of the complexed form from the uncomplexed form of one or both of the interacting proteins, as well as to accommodate automation of the assay. Binding of a test compound to the IL-23 receptor protein or interaction of the cytokine receptor protein with a target molecule (e.g., IL-23 or one of the peptides or peptide derivatives described herein) in the presence and absence of a test compound, can be accomplished in any vessel suitable for containing the reactants. Examples of such vessels include microtitre plates, test tubes, and micro-centrifuge tubes.

Further, a fusion protein may be provided which adds a domain that allows one or both of the proteins to be bound to a matrix. For example: glutathione-S-transferase/IL-23 receptor fusion proteins or glutathione-S-transferase/IL-23 receptor fusion proteins can be adsorbed onto glutathione sepharose beads (Sigma Chemical, St. Louis, Mo.), or glutathione derivatized microtitre plates, which are then combined with the test compound or the test compound and either the non-adsorbed target protein or IL-23 receptor protein and the mixture incubated under conditions conducive to complex formation (e.g., at physiological conditions for salt and pH). Following incubation the beads or microtitre plate wells are washed to remove any unbound components, and complex formation determined either directly or indirectly, for example, as described herein. Alternatively, the complexes can be dissociated from the matrix, and the level of IL-23 receptor binding or activity determined using standard techniques.

Other techniques for immobilizing proteins on matrices (well-known in the art) can also be used in the screening assays of the invention. For example, either a IL-23 receptor protein or a molecule that interacts with the IL-23 receptor can be immobilized by conjugation of biotin and streptavidin. Biotinylated IL-23 receptor protein or IL-23 receptor interacting molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96-well plates (Pierce Chemical). Alternatively, antibodies reactive with the IL-23 receptor protein or IL-23 receptor interacting molecules, but which do not interfere with binding of the IL-23 receptor protein to its interacting molecule, can be adhered to the wells of the plate, and unbound target or cytokine receptor protein trapped in the wells by antibody conjugation. Methods for detecting such complexes, in addition to those described above for the GST-immobilized complexes, include immunodetection of complexes using antibodies reactive with the cytokine receptor protein or target molecule, as well as enzyme-linked assays which rely on detecting an enzymatic activity associated with the IL-23 receptor or IL-23 receptor interacting molecule.

It shall be understood that the in vivo experimental model can also be used to carry out an in vitro assay.

### In Vitro Assays

Candidate peptides may be tested for their ability to modulate the phosphorylation state of IL-23 receptor protein or portion thereof, or an upstream or downstream target protein, using, for example, an in vitro kinase assay. Briefly, an IL-23 receptor target molecule (e.g., an immunoprecipitated receptor from a cell line expressing such a molecule), can be incubated with radioactive ATP, e.g., gamma-³²P-ATP, in a buffer containing MgCl₂ and MnCl₂, e.g., 10 mM MgCl₂ and 5 mM MnCl₂. Following the incubation, the immunoprecipitated receptor target molecule, can be separated by sodium dodecyl sulfate (SDS)-polyacrylamide gel electrophoresis under reducing conditions, transferred to a membrane, e.g., a polyvinylidene difluoride (PVDF) membrane, and autoradiographed. The appearance of detectable bands on the autoradiograph indicates that the receptor substrate has been phosphorylated. Phosphoaminoacid analysis of the phosphorylated substrate can also be performed to determine which residues on the receptor substrate are phosphorylated. Briefly, the radiophosphorylated protein band can be excised from the SDS gel and subjected to partial acid hydrolysis. The products can then be separated by one-dimensional electrophoresis and analyzed on, for example, a phosphoimager and compared to ninhydrin-stained phosphoaminoacid standards. Such assays are described in, for example, Tamaskovic et al. (Biol. Chem. 380(5):569-78, 1999).

In particular, candidate peptides targeting IL-23 receptor may be tested by measuring IL-23 induced TNFα or STAT3 phosphorylation in activated macrophages (e.g., HL60 cells), in splenocytes, or in TH17 cells (Streeck et al., J. Immunol. Methods 333:115-120, 2008; Kebir et al., Nat. Med. 13:1173-1175, 2007) as described herein.

### In Vivo Assays

The assays described above may be used as initial or primary screens to detect promising lead compounds for further development. Lead peptides can be further assessed in additional, secondary screens, which may involve various assays utilizing activated macrophages or natural killer cell lines expressing these receptors or other assays.

Tertiary screens may involve the study of the identified inhibitors or stimulators in animal models for clinical symptoms. Thus, a compound (e.g., a peptide or peptidomimetic) identified as described herein desirably is also tested in an appropriate animal model such as a rat or a mouse. For example, a peptide can be used in an animal model to determine the efficacy, toxicity, or side effects of treatment with such an agent. Alternatively, an agent identified as described herein can be used in an animal model to determine the mechanism of action of such an agent. Furthermore, the present invention includes uses of novel agents identified by the above-described screening assays for treatment (e.g., treatment of an autoimmune or inflammatory disorder), as described herein. Non-limiting animal models which can be used in such assays include: inflammatory bowel disease (IBD), experimental autoimmune encephalitis (EAE), and psoriatic dermatitis models. Such models are standard in the art and are described herein.

### Peptide Preparation

The peptides or peptide derivatives of the present invention may be obtained by any method of peptide synthesis known to those skilled in the art, including synthetic (e.g., exclusive solid phase synthesis, partial solid phase synthesis, fragment condensation, classical solution synthesis, native-chemical ligation) and recombinant techniques. For example, the peptides or peptides derivatives can be obtained by solid phase peptide synthesis, which in brief, consist of coupling the carboxyl group of the C-terminal amino acid to a resin (e.g., benzhydrylamine resin, chloromethylated resin, hydroxymethyl resin) and successively adding N-alpha protected amino acids. The protecting groups may be any such groups known in the art. Before each new amino acid is added to the growing chain, the protecting group of the previous amino acid added to the chain is removed. Such solid phase synthesis has been described, for example, by Merrifield, (J. Am. Chem. Soc. 85: 2149 (1964)); Vale et al., (Science 213:1394-1397 (1981)), in U.S. Pat. Nos. 4,305,872 and 4,316,891, Bodonsky et al. (Chem. Ind. (London), 38:1597 (1966)); and Pieta and Marshall, (Chem. Comm. 650 (1970)) by techniques reviewed in Lubell et al. (“Peptides.”Science of Synthesis 21.11, Chemistry of Amides. Thieme, Stuttgart, 713-809 (2005)). The coupling of amino acids to appropriate resins is also well known in the art and has been described in U.S. Pat. No. 4,244,946. (Reviewed in Houver-Weyl, Methods of Organic Chemistry. Vol E22a. Synthesis of Peptides and Peptidomimetics, Murray Goodman, Editor-in-Chief, Thieme. Stuttgart. New York 2002).

During any process of the preparation of the compound of the present invention, it may be necessary and/or desirable to protect sensitive reactive groups on any of the molecule concerned. This may be achieved by means of conventional protecting groups such as those described in Protective Groups In Organic Synthesis by T. W. Greene & P. G. M. Wuts, 1991, John Wiley and Sons, New-York; and Peptides: chemistry and Biology by Sewald and Jakubke, 2002, Wiley-VCH, Wheinheim p. 142. For example, alpha amino protecting groups include acyl type protecting groups (e.g., trifluoroacetyl, formyl, acetyl), aliphatic urethane protecting groups (e.g., t-butyloxycarbonyl (BOC), cyclohexyloxycarbonyl), aromatic urethane type protecting groups (e.g., fluorenyl-9-methoxy-carbonyl (Fmoc), benzyloxycarbonyl (Cbz), Cbz derivatives) and alkyl type protecting groups (e.g., triphenyl methyl, benzyl). The amino acids side chain protecting groups include benzyl (for Thr and Ser), Cbz (Tyr, Thr, Ser, Arg, Lys), methyl ethyl, cyclohexyl (Asp, His), Boc (Arg, His, Cys) etc. The protecting groups may be removed at a convenient subsequent stage using methods known in the art.

Further, the peptides of the present invention, including the analogs and other modified variants, may be synthesized according to the FMOC protocol in an organic phase with protective groups. Desirably, the peptides are purified with a yield of 70% with high-pressure liquid chromatography (HPLC) on a C18 chromatography column and eluted with an acetonitrile gradient of 10-60%. The molecular weight of a peptide can be verified by mass spectrometry (reviewed in Fields, G. B. “Solid-Phase Peptide Synthesis” Methods in Enzymology. Vol. 289, Academic Press, 1997).

Alternatively, peptides of the present invention may be prepared in recombinant systems using, for example, polynucleotide sequences encoding the peptides. It is understood that a peptide may contain more than one of the above-described modifications within the same peptide. Also included in the present invention are pharmaceutically acceptable salt complexes of the peptides of described herein or their derivatives.

Purification of the synthesized peptides or peptide derivatives may be carried out by standard methods, including chromatography (e.g., ion exchange, size exclusion, and affinity), centrifugation, precipitation or any standard technique for the purification of peptides and peptides derivatives. For example, thin-layered chromatography or reverse phase HPLC may be employed. Other purification techniques well known in the art and suitable for peptide isolation and purification may also be used.

Where the processes for the preparation of the compounds according to the present invention give rise to mixtures of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their components enantiomers by standard techniques such as the formation of diastereoisomeric pairs by salt formation with an optically active acid followed by fractional crystallization and regeneration of the free base. The compounds may also be resolved by formation of diastereomeric esters or amides, followed by removal of the chiral auxiliary. Alternatively, the compounds may be resolved using a chiral HPLC column.

### Preparation of Peptide Derivatives and Peptidomimetics

In addition to peptides consisting only of naturally occurring amino acids, peptidomimetics or peptide analogs are also encompassed by the present invention. Peptide analogs are commonly used in the pharmaceutical industry as non-peptide drugs with properties analogous to those of the template peptide. The non-peptide compounds are termed “peptide mimetics” or peptidomimetics (Fauchere et al., Infect. Immun. 54:283-287 (1986); Evans et al., J. Med. Chem. 30:1229-1239 (1987)). Peptide mimetics that are structurally related to therapeutically useful peptides may be used to produce an equivalent or enhanced therapeutic or prophylactic effect. Generally, peptidomimetics are structurally similar to the paradigm polypeptide (i.e., a polypeptide that has a biological or pharmacological activity) such as naturally-occurring receptor-binding polypeptides, but have one or more peptide linkages optionally replaced by linkages such as —CH₂NH—, —CH₂S—, —CH₂CH₂—, —CH═CH— (cis and trans), —CH₂SO—, —CH(OH)CH₂—, —COCH₂— etc., by methods well known in the art (Spatola, Peptide Backbone Modifications, Vega Data, 1(3):267 (1983)); Spatola et al. (Life Sci. 38:1243-1249 (1986)); Hudson et al. (Int. J. Pept. Res. 14:177-185 (1979)); and Weinstein. B., 1983, Chemistry and Biochemistry, of Amino Acids, Peptides and Proteins, Weinstein eds, Marcel Dekker, New-York). Such peptide mimetics may have significant advantages over naturally-occurring polypeptides including more economical production, greater chemical stability, enhanced pharmacological properties (e.g., half-life, absorption, potency, efficiency, etc), reduced antigenicity and others.

While peptides may be effective in inhibiting a biological activity of a receptor (e.g., wild-type IL-23R) in vitro, their effectiveness in vivo might be reduced by the presence of proteases. Serum proteases have specific substrate requirements. The substrate must have both L-amino acids and peptide bonds for cleavage. Furthermore, exopeptidases, which represent the most prominent component of the protease activity in serum, usually act on the first peptide bond of the peptide and require a free N-terminus (Powell et al., Pharm. Res. 10:1268-1273 (1993)). In light of this, it is often advantageous to use modified versions of peptides. The modified peptides retain the structural characteristics of the original L-amino acid peptides that confer biological activity with regard to IL-23R but are advantageously not readily susceptible to cleavage by protease and/or exopeptidases.

Systematic substitution of one or more amino acids of a consensus sequence with D-amino acid of the same type (e.g., D-lysine in place of L-lysine) may be used to generate more stable peptides. Thus, a peptide derivative or peptidomimetic of the present invention may be all L, all D or mixed D, L peptide, in either forward or reverse order. The presence of an N-terminal or C-terminal D-amino acid increases the in vivo stability of a peptide since peptidases cannot utilize a D-amino acid as a substrate (Powell et al., Pharm. Res. 10:1268-1273 (1993)). Reverse-D peptides are peptides containing D-amino acids, arranged in a reverse sequence relative to a peptide containing L-amino acids. Thus, the C-terminal residue of an L-amino acid peptide becomes N-terminal for the D-amino acid peptide, and so forth. Reverse D-peptides retain the same tertiary conformation and therefore the same activity, as the L-amino acid peptides, but are more stable to enzymatic degradation in vitro and in vivo, and thus have greater therapeutic efficacy than the original peptide (Brady and Dodson, Nature 368:692-693 (1994); Jameson et al., Nature 368:744-746 (1994)). Similarly, a reverse-L peptide may be generated using standard methods where the C-terminus of the parent peptide is now the N-terminus of the reverse-L peptide. Moreover, a reverse peptide may contain a combination of L- and D-amino acids.

In addition, constrained peptides comprising a consensus sequence or a substantially identical consensus sequence variation may be generated by methods well known in the art (Rizo and Gierasch, Ann. Rev. Biochem. 61:387-418 (1992)). For example, constrained peptides may be generated by adding cysteine residues capable of forming disulfide bridges and, thereby, resulting in a cyclic peptide. Cyclic peptides have no free N- or C-termini. Accordingly, they are not susceptible to proteolysis by exopeptidases, although they are, of course, susceptible to endopeptidases, which do not cleave at peptide termini. The amino acid sequences of the peptides with N-terminal or C-terminal D-amino acids and of the cyclic peptides are usually identical to the sequences of the peptides to which they correspond, except for the presence of N-terminal or C-terminal D-amino acid residue, or their circular structure, respectively.

A cyclic derivative containing an intramolecular disulfide bond may be prepared by conventional solid phase synthesis while incorporating suitable S-protected cysteine or homocysteine residues at the positions selected for cyclization such as the amino and carboxy termini (Sah et al., J. Pharm. Pharmacol. 48:197 (1996)). Following completion of the chain assembly, cyclization can be performed either (1) by selective removal of the S-protecting group with a consequent on-support oxidation of the corresponding two free SH-functions, to form a S—S bonds, followed by conventional removal of the product from the support and appropriate purification procedure or (2) by removal of the peptide from the support along with complete side chain de-protection, followed by oxidation of the free SH-functions in highly dilute aqueous solution.

The cyclic derivative containing an intramolecular amide bond may be prepared by conventional solid phase synthesis while incorporating suitable amino and carboxyl side chain protected amino acid derivatives, at the position selected for cyclization. The cyclic derivatives containing intramolecular —S-alkyl bonds can be prepared by conventional solid phase chemistry while incorporating an amino acid residue with a suitable amino-protected side chain, and a suitable S-protected cysteine or homocysteine residue at the position selected for cyclization.

Substitution of non-naturally-occurring amino acids for natural amino acids in a subsequence of the peptides can also confer resistance to proteolysis. Such a substitution can, for instance, confer resistance to proteolysis by exopeptidases acting on the N-terminus without affecting biological activity. Examples of non-naturally-occurring amino acids include α,α-disubstituted amino acids, N-alkyl amino acids, lactic acids, C-α-methyl amino acids, β-amino acids, and β-methyl amino acids. Amino acids analogs useful in the present invention may include, but are not limited to, β-alanine, norvaline, norleucine, 4-aminobutyric acid, orithine, hydroxyproline, sarcosine, citrulline, cysteic acid, cyclohexylalanine, 2-aminoisobutyric acid, 6-aminohexanoic acid, t-butylglycine, phenylglycine, o-phosphoserine, N-acetyl serine, N-formylmethionine, 3-methylhistidine and other unconventional amino acids. Furthermore, the synthesis of peptides with non-naturally-occurring amino acids is routine in the art.

Another effective approach to confer resistance to peptidases acting on the N-terminal or C-terminal residues of a peptide is to add chemical groups at the peptide termini, such that the modified peptide is no longer a substrate for the peptidase. One such chemical modification is glycosylation of the peptides at either or both termini. Certain chemical modifications, in particular N-terminal glycosylation, have been shown to increase the stability of peptides in human serum (Powell et al., Pharm. Res. 10:1268-1273 (1993)). Other chemical modifications which enhance serum stability include, but are not limited to, the addition of an N-terminal alkyl group, consisting of a lower alkyl of from one to twenty carbons, such as an acetyl group, and/or the addition of a C-terminal amide or substituted amide group. In particular, the present invention includes modified peptides consisting of peptides bearing an N-terminal acetyl group and/or a C-terminal amide group.

Also included by the present invention are other types of peptide derivatives containing additional chemical moieties not normally part of the peptide, provided that the derivative retains the desired functional activity of the peptide. Examples of such derivatives include (1) N-acyl derivatives of the amino terminal or of another free amino group, wherein the acyl group may be an alkanoyl group (e.g., acetyl, hexanoyl, octanoyl) an aroyl group (e.g., benzoyl) or a blocking group such as F-moc (fluorenylmethyl-O—CO—); (2) esters of the carboxy terminal or of another free carboxy or hydroxyl group; (3) amide of the carboxy-terminal or of another free carboxyl group produced by reaction with ammonia or with a suitable amine; (4) phosphorylated derivatives; (5) derivatives conjugated to an antibody or other biological ligand and other types of derivatives; and (6) derivatives conjugated to a polyethylene glycol (PEG) chain.

Longer peptide sequences which result from the addition of additional amino acid residues to the peptides of the invention are also encompassed in the present invention. Such longer peptide sequence would be expected to have the same biological activity (e.g., inhibiting activation of an IL-23 receptor) as the peptides described above. While peptides having a substantial number of additional amino acids are not excluded, it is recognized that some large polypeptides may assume a configuration that masks the effective sequence, thereby preventing binding to, for example, the IL-23 receptor. These derivatives could act as competitive antagonists. Thus, while the present invention encompasses peptides or derivatives of the peptides described herein having an extension, desirably the extension does not destroy the IL-23 receptor modulating activity of the peptide or derivative.

Other derivatives included in the present invention are dual peptides consisting of two of the same, or two different peptides of the present invention covalently linked to one another either directly or through a spacer, such as by a short stretch of alanine residues or by a putative site for proteolysis (e.g., by cathepsin, see e.g., U.S. Pat. No. 5,126,249 and European Patent Number 495 049). Multimers of the peptides of the present invention consist of polymer of molecules formed from the same or different peptides or derivatives thereof.

The present invention also encompasses peptide derivatives that are chimeric or fusion proteins containing a peptide described herein, or fragment thereof, linked at its amino- or carboxy-terminal end, or both, to an amino acid sequence of a different protein. Such a chimeric or fusion protein may be produced by recombinant expression of a nucleic acid encoding the protein. For example, a chimeric or fusion protein may contain at least 6 amino acids of a peptide of the present invention and desirably has a functional activity equivalent or greater than a peptide of the invention.

Peptide derivatives of the present invention can be made by altering the amino acid sequences by substitution, addition, or deletion or an amino acid residue to provide a functionally equivalent molecule, or functionally enhanced or diminished molecule, as desired. The derivative of the present invention include, but are not limited to, those containing, as primary amino acid sequence, all or part of the amino acid sequence of the peptides described herein (e.g., a 2301, 2303, 2305, 2307, or 2309 peptide) including altered sequences containing substitutions of functionally equivalent amino acid residues. For example, one or more amino acid residues within the sequence can be substituted by another amino acid of a similar polarity, which acts as a functional equivalent, resulting in a silent alteration. Substitution for an amino acid within the sequence may be selected from other members of the class to which the amino acid belongs. For example, the positively charged (basic) amino acids include arginine, lysine, and histidine. The nonpolar (hydrophobic) amino acids include leucine, isoleucine, alanine, phenylalanine, valine, proline, tryptophane, and methionine. The uncharged polar amino acids include serine, threonine, cysteine, tyrosine, asparagine, and glutamine. The negatively charged (acid) amino acids include glutamic acid and aspartic acid. The amino acid glycine may be included in either the nonpolar amino acid family or the uncharged (neutral) polar amino acid family. Substitutions made within a family of amino acids are generally understood to be conservative substitutions.

### Assays to Identify Peptidomimetics

As described above, non-peptidyl compounds generated to replicate the backbone geometry and pharmacophore display (peptidomimetics) of the peptides identified by the methods of the present invention often possess attributes of greater metabolic stability, higher potency, longer duration of action and better bioavailability.

The peptidomimetics compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, Anticancer Drug Des. 12:145 (1997)). Examples of methods for the synthesis of molecular libraries can be found in the art, for example, in: DeWitt et al. (Proc. Natl. Acad. Sci. USA 90:6909 (1993)); Erb et al. (Proc. Natl. Acad. Sci. USA 91:11422 (1994)); Zuckermann et al. (J. Med. Chem. 37:2678 (1994)); Cho et al. (Science 261:1303 (1993)); Carell et al. (Angew. Chem., Int. Ed. Engl. 33:2059 (1994) and ibid 2061); and in Gallop et al. (Med. Chem. 37:1233 (1994)). Libraries of compounds may be presented in solution (e.g., Houghten, Biotechniques 13:412-421 (1992)) or on beads (Lam, Nature 354:82-84 (1991)), chips (Fodor, Nature 364:555-556 (1993)), bacteria or spores (U.S. Pat. No. 5,223,409), plasmids (Cull et al., Proc. Natl. Acad. Sci. USA 89:1865-1869 (1992)) or on phage (Scott and Smith, Science 249:386-390 (1990)), or luciferase, and the enzymatic label detected by determination of conversion of an appropriate substrate to product.

Once a peptide of the present invention is identified, it may be isolated and purified by any number of standard methods including, but not limited to, differential solubility (e.g., precipitation), centrifugation, chromatography (e.g., affinity, ion exchange, size exclusion, and the like) or by any other standard techniques used for the purification of peptides, peptidomimetics or proteins. The functional properties of an identified peptide of interest may be evaluated using any functional assay known in the art. Desirably, assays for evaluating downstream receptor function in intracellular signaling are used (e.g., cell proliferation).

For example, the peptidomimetics compounds of the present invention may be obtained using the following three-phase process: (1) scanning the peptides of the present invention to identify regions of secondary structure necessary for recognition and activity toward the IL-23 receptor; (2) using conformationally constrained dipeptide surrogates to refine the backbone geometry and provide organic platforms corresponding to these surrogates; and (3) using the best organic platforms to display organic pharmocophores in libraries of candidates designed to mimic the desired activity of the native peptide. In more detail the three phases are as follows. In phase 1, the lead candidate peptides are scanned and their structure abridged to identify the requirements for their activity. A series of peptide analogs of the original are synthesized. In phase 2, the best peptide analogs are investigated using the conformationally constrained dipeptide surrogates. Indolizidin-2-one, indolizidin-9-one, and quinolizidinone amino acids (I²aa, I⁹aa and Qaa respectively) are used as platforms for studying backbone geometry of the best peptide candidates. These and related platforms (reviewed in Halab et al., Biopolymers 55:101-122 (2000); and Hanessian et al. Tetrahedron 53:12789-12854 (1997)) may be introduced at specific regions of the peptide to orient the pharmacophores in different directions. Biological evaluation of these analogs identifies improved lead peptides that mimic the geometric requirements for activity. In phase 3, the platforms from the most active lead peptides are used to display organic surrogates of the pharmacophores responsible for activity of the native peptide. The pharmacophores and scaffolds are combined in a parallel synthesis format. Derivation of peptides and the above phases can be accomplished by other means using methods known in the art.

Structure function relationships determined from the peptides, peptide derivatives, peptidomimetics or other small molecules of the present invention may be used to refine and prepare analogous molecular structures having similar or better properties. Accordingly, the compounds of the present invention also include molecules that share the structure, polarity, charge characteristics and side chain properties of the peptides described herein.

In summary, based on the disclosure herein, those skilled in the art can develop peptides and peptidomimetics screening assays which are useful for identifying compounds for inhibiting cytokine receptor activity. Compounds so identified may also be shown to activate these receptors. The assays of this invention may be developed for low-throughput, high-throughput, or ultra-high throughput screening formats. Assays of the present invention include assays which are amenable to automation.

### Pharmaceutical Compositions

The peptides, peptide derivatives and peptidomimetics of the present invention are useful in the treatment of conditions or diseases associated with a biological activity of an IL-23 receptor, such as an autoimmune or inflammatory disorder. Generally, such treatments involve administering to a subject in need thereof an effective amount of a peptide, peptide derivative or peptidomimetic, or a composition comprising a peptide, peptide derivative or peptidomimetic to inhibit a IL-23 receptor activity. For example, an effective amount of a therapeutic composition containing a peptide (e.g., a 2305, 2307, 2309, or 2303 peptide or a derivative thereof as set forth in Formula I, Formula II, Formula III, or Formula IV) and a suitable pharmaceutical carrier may be administered to a subject to inhibit a biological activity of the IL-23 receptor targeted by the peptide to prevent, ameliorate symptoms or treat a disorder, disease or condition related to abnormal signaling through the cytokine receptor (e.g., overstimulation of the IL-23 receptor via an overproduction of IL-23 ligand or via a constitutively active receptor or any other defect). The subject desirably is a mammal (e.g., a human).

The peptides, peptide derivatives and peptidomimetics of the present invention may be used in the treatment, prophylaxy or amelioration of symptoms in any disease condition or disorder where the inhibition of IL-23 receptor biological activity might be beneficial. Such diseases, conditions or disorders include, but are not limited to, inflammatory or autoimmune disorders such as inflammatory bowel disease, psoriasis, and multiple sclerosis.

The pharmaceutical compositions can be in a variety of forms including oral dosage forms, topic creams, suppository, nasal spray and inhaler, as well as injectable and infusible solutions. Methods for preparing pharmaceutical composition are well known in the art.

Compositions within the scope of the present invention desirably contain the active agent (e.g. peptide, peptide derivative or peptidomimetics) in an amount effective to achieve the desired therapeutic effect while avoiding adverse side effects. Pharmaceutically acceptable preparations and salts of the active agent are within the scope of the present invention and are well known in the art. For the administration of polypeptide antagonists and the like, the amount administered desirably is chosen so as to avoid adverse side effects. The amount of the therapeutic or pharmaceutical composition which is effective in the treatment of a particular disease, disorder or condition depends on the nature and severity of the disease, the target site of action, the subject's weight, special diets being followed by the subject, concurrent medications being used, the administration route and other factors that are recognized by those skilled in the art. The dosage can be adapted by the clinician in accordance with conventional factors such as the extent of the disease and different parameters from the subject. Typically, 0.001 to 100 mg/kg/day is administered to the subject. Effective doses may be extrapolated from dose response curves derived from in vitro or animal model test systems. For example, in order to obtain an effective mg/kg dose for humans based on data generated from rat studies, the effective mg/kg dosage in rat is divided by six.

Various delivery systems are known and can be used to administer peptides, peptide derivatives or peptidomimetics or a pharmaceutical composition of the present invention. The pharmaceutical composition of the present invention can be administered by any suitable route including, intravenous or intramuscular injection, intraventricular or intrathecal injection (for central nervous system administration), orally, topically, subcutaneously, subconjunctivally, or via intranasal, intradermal, sublingual, vaginal, rectal or epidural routes.

Other delivery system well known in the art can be used for delivery of the pharmaceutical compositions of the present invention, for example via aqueous solutions, encapsulation in microparticules, or microcapsules.

The pharmaceutical compositions of the present invention can also be delivered in a controlled release system. For example, a polymeric material can be used (see, e.g., Smolen and Ball, Controlled Drug Bioavailability, Drug product design and performance, 1984, John Wiley & Sons; Ranade and Hollinger, Drug Delivery Systems, pharmacology and toxicology series, 2003, 2^{nd} edition, CRRC Press). Alternatively, a pump may be used (Saudek et al., N. Engl. J. Med. 321:574 (1989)).

Compounds of the present invention may also be delivered by the use of monoclonal antibodies as individual carriers to which the compound molecules are coupled. The compounds of the present invention may also be coupled to a class of biodegradable polymers useful in achieving controlled release of the drug, for example, polylactic acid, polyorthoesters, cross-linked amphipathic block copolymers and hydrogels, polyhydroxy butyric acid, and polydihydropyrans.

As described above, pharmaceutical compositions of the present invention desirably include a peptide, peptide derivatives or peptidomimetic combined with a pharmaceutically acceptable carrier. The term carrier refers to diluents, adjuvants, excipients or vehicles with which the peptide, peptide derivative or peptidomimetic is administered. Such pharmaceutical carriers include sterile liquids such as water and oils including mineral oil, vegetable oil (e.g., soybean oil or corn oil), animal oil or oil of synthetic origin. Aqueous glycerol and dextrose solutions as well as saline solutions may also be employed as liquid carriers of the pharmaceutical compositions of the present invention. The choice of the carrier depends on factors well recognized in the art, such as the nature of the peptide, peptide derivative or peptidomimetic, its solubility and other physiological properties as well as the target site of delivery and application. For example, carriers that can penetrate the blood brain barrier are used for treatment, prophylaxis or amelioration of symptoms of diseases or conditions (e.g. inflammation or an autoimmune disorder) in the central nervous system. Examples of suitable pharmaceutical carriers are described in Remington: The Science and Practice of Pharmacy by Alfonso R. Gennaro, 2003, 21^{th} edition, Mack Publishing Company. Moreover, suitable carriers for oral administration are known in the art and are described, for example, in U.S. Pat. Nos. 6,086,918, 6,673,574, 6,960,355, and 7,351,741 and in WO2007/131286, the disclosures of which are hereby incorporated by reference.

Further pharmaceutically suitable materials that may be incorporated in pharmaceutical preparations of the present invention include absorption enhancers, pH regulators and buffers, osmolarity adjusters, preservatives, stabilizers, antioxidants, surfactants, thickeners, emollient, dispersing agents, flavoring agents, coloring agents, and wetting agents.

Examples of suitable pharmaceutical excipients include, water, glucose, sucrose, lactose, glycol, ethanol, glycerol monostearate, gelatin, starch flour (e.g., rice flour), chalk, sodium stearate, malt, sodium chloride, and the like. The pharmaceutical compositions of the present invention can take the form of solutions, capsules, tablets, creams, gels, powders sustained release formulations and the like. The composition can be formulated as a suppository, with traditional binders and carriers such as triglycerides (see Remington: The Science and Practice of Pharmacy by Alfonso R. Gennaro, 2003, 21^{th} edition, Mack Publishing Company). Such compositions contain a therapeutically effective amount of the therapeutic composition, together with a suitable amount of carrier so as to provide the form for proper administration to the subject. The formulations are designed to suit the mode of administration and the target site of action (e.g., a particular organ or cell type).

The pharmaceutical compositions of the present invention can be formulated as neutral or salt forms. Pharmaceutically acceptable salts include those that form with free amino groups and those that react with free carboxyl groups. Non-toxic alkali metal, alkaline earth metal, and ammonium salts commonly used in the pharmaceutical industry include sodium, potassium, lithium, calcium, magnesium, barium, ammonium, and protamine zinc salts, which are prepared by methods well known in the art. Also included are non-toxic acid addition salts, which are generally prepared by reacting the compounds of the present invention with suitable organic or inorganic acid. Representative salts include the hydrobromide, hydrochloride, valerate, oxalate, oleate, laureate, borate, benzoate, sulfate, bisulfate, acetate, phosphate, tysolate, citrate, maleate, fumarate, tartrate, succinate, napsylate salts, and the like.

The present invention also provides for modifications of peptides or peptide derivatives such that they are more stable once administered to a subject (i.e., once administered it has a longer half-life or longer period of effectiveness as compared to the unmodified form). Such modifications are well known to those skilled in the art to which this invention pertain (e.g., polyethylene glycol derivatization a.k.a. PEGylation, microencapsulation, etc).

The IL-23 receptor antagonists of the present invention may be administered alone or in combination with other active agents (e.g., an anti-inflammatory compound) useful for the treatment, prophylaxis or amelioration of symptoms of a IL-23 receptor associated disease or condition. Thus, the compositions and methods of the present invention can be used in combination with other agents exhibiting the ability to modulate IL-23 activity (e.g., synthesis, release and/or binding to the IL-23 receptor) or to reduce the symptoms of the IL-23 receptor associated disease (e.g., an autoimmune or inflammatory disorder). Examples of such agents include, but are not limited to, monoclonal antibodies (Pfizer, CP-751,871; Imclone, IMC-A12; Merck 7C10; Schering-Plough, 19D12) or tyrosine kinase inhibitors (Insmed, INSM18 PPP; Biovitrium, Karolinska Institute (Girnita et al., 2004; Vasilcanu et al., 2004); NVP-ADW742, AEW541, Novartis (Mitsiades CS, 2004); BMS-536924, BMS-554417, Bristol-Myers Squibb).

The present invention is illustrated in further detail by the following non-limiting examples. The examples are provided for illustration only and should not be construed as limiting the scope of the invention.

### EXAMPLES

### Example 1

### Identification of IL-23R Antagonists and Agonists

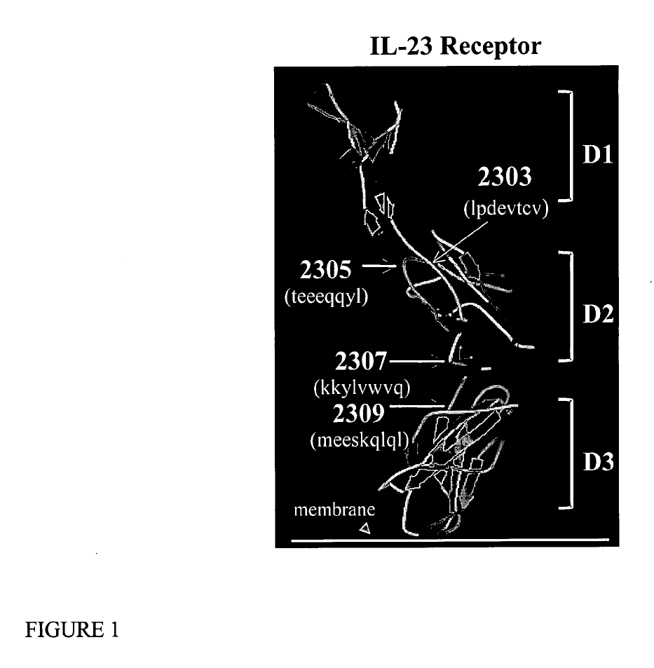

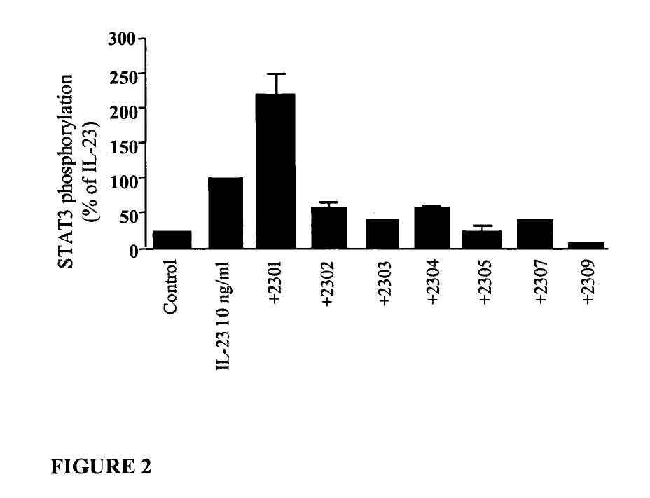

The domain similarities (IgG-like domains) were determined with ProDom (Boeckmann et al., Nucleic Ac Res 31:365-370 (2003)), PROSITE (Rost, Enzymol 266:525-539 (1996)) and Predict Protein (Rost, Enzymol 266:525-539 (1996)) to confirm the position of the different regions of the IL-23 receptor and the secondary structure distribution. Hydrophobic and flexibility profiles were then examined with the program ProtScale (Kyte et al., J Mol Biol 157:105-132, 1982). As described above, IL-23R and IL-12R are structurally similar as they share common ligand and receptor subunits. Maintenance of integrity of IL-12R activation is desirable to preserve immunocompetence (Kenakin, Mol. Intervention. 4:222-229 (2004); Langrish et al., Immunol Rev 202:96-105 (2004)). However, IL-12Rβ2 and IL-23R only exhibit 24% similarities. Hence, based on crystallography, molecular modeling, and hydropathy profile, extracellular regions specific to IL-23R (identified in depicted model of IL-23R; FIG. 1) were targeted by designing small peptides reproducing these sequences. A prerequisite sequence homology analysis (Blast analysis; NCBI) of these peptides was performed to ascertain that the sequences are unique to IL-23R. Screening of the initial focused IL-23R peptide library in IL-23-induced STAT3 phosphorylation identified 4 compounds with demonstrable efficacy (‘hits’) which were named 2303, 2305, 2307, and 2309 (FIG. 2). In addition, an agonist of IL-23R biological activity was identified and named 2301 (DLSSGYPPDI; SEQ ID NO:5).

### Example 2

### Characterization of IL-23R Antagonists

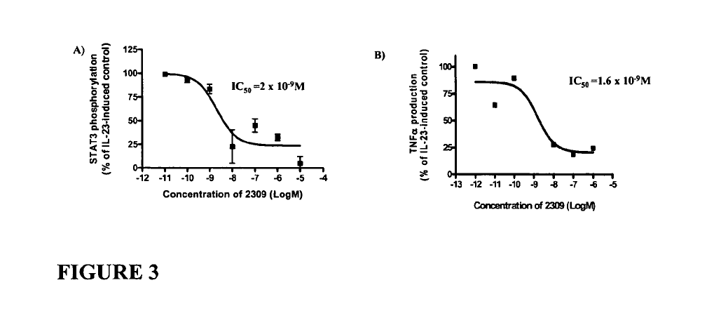

D-peptides 2303, 2305, 2307 and 2309 (1 μM) inhibit IL-23-induced STAT3 phosphorylation in phorbol 12-myristate 13-acetate (PMA)-activated human monocytes (HL-60) (FIG. 2). Because of reproducibility of results, concentration-response to 2309 was studied further on the same assay and revealed Emax≈85% and IC₅₀=2 nM (FIG. 3A). This efficacy was further corroborated using a separate outcome parameter, specifically tumor necrosis factor (TNF) formation (measured by enzyme-linked immunosorbent assay (ELISA); FIG. 3B), which yielded equivalent Emax≈80% and IC₅₀=1.6 nM.

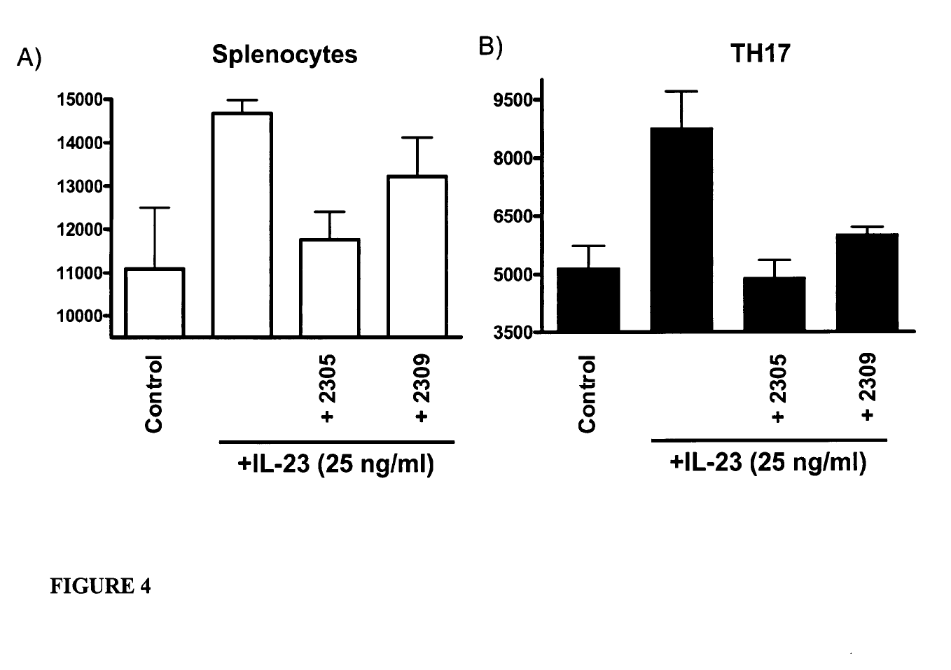

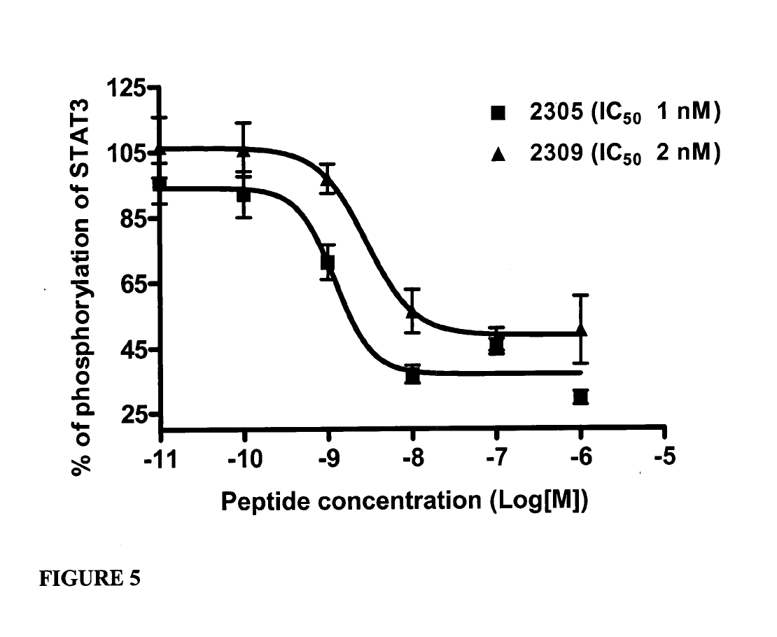

In splenocytes and TH17 cells, respectively, peptide APG-2305 (1 μM) inhibited 75% and 100% of IL-23-induced STAT3 phosphorylation while peptide APG-2309 (1 μM) inhibited 50% and 75% of IL-23-induced-STAT3 phosphorylation (25 ng/ml)(FIGS. 4, 6, and 7). Peptides APG-2305 and APG-2309 showed potencies (IC50s) of 1 nM and 2 nM (FIG. 5). As noted above, peptide 2305 is derived from a loop in the second domain of IL-23R subunit that interacts with the inter-region between D1 and D2 domains and peptide 2309 is derived from a loop in the D3 domain of IL-23R (see FIG. 1). Both peptides inhibit IL-23-induced phosphorylation but to a different extent. These peptides interact in different regions of the receptor or different regions of dimerization of the IL-23R subunit with IL-12b1 and therefore may have a different effect on the biological activity of IL-23R by inducing different conformational changes.

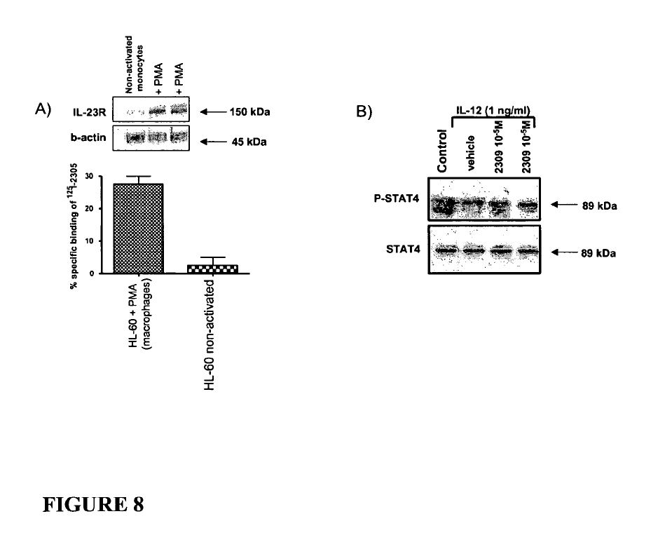

Peptide 2305 specifically bound to IL-23R-expressing phorbol 12-myristate 13-acetate (PMA)-activated monocytes, but not to IL-23R-devoid monocytes (FIG. 8A). Consistent with these observations, the efficacy of peptide 2305 was shown in IL-23R expressing PMA-activated human monocytes, but not in native (non-activated) monocytes (see FIG. 8A).

Selectivity of peptide 2309 towards IL-23 was demonstrated by the inability of peptide 2309 (even at high concentrations, i.e., 10 μM) to interfere with IL-12-induced STAT4 phosphorylation (FIG. 8B). Peptide 2309 remained capable of inhibiting 11,23-induced STAT3 phosphorylation (FIG. 2).

The experiments described herein were carried out using the following materials and methods.

Isolation of Splenocytes

Spleens were sterilely removed from mice, sliced, and mashed in the presence of PBS buffer, 2% fetal bovine serum, and 1 mM EDTA. Tissue was allowed to pass through a number 26 syringe and was filtered through a 70 μm mesh nylon strainer. Cells were cultured with non-essential amino acids, 2 μg/ml of anti-CD3, and 20 μg/ml of anti-CD-28 as survival and differentiation factors.

Isolation of CD4+ Cells and Differentiation in TH17 Cells

CD4+ cells were isolated from 2.5×10⁸ splenocytes with the EasySep isolation kit (StemCell Technologies) according to the manufacturer's instructions. CD4+ cells cells were incubated overnight with non-essential amino acids and anti-CD3 and CD28 antibodies (see above). The next day the cells were split into 12-well plates (1-2×10⁶ cells/well) and incubated with complete RPMI medium (10% FBS, Pen/Strep, and non-essential amino acids (1×)) and a differentiation cocktail containing 2 μg/ml of anti-CD3, 2 μg/ml of anti-CD28, 5 ng/ml of TGFb1, 20 ng/ml of IL-6, 10 ng/ml of IL-23, 2 μg/ml of anti-rat anti IFNγ and 10 μg/ml of anti-mouse IL-4 (final concentrations).

Determination of STAT3 Phosphorylation

Splenocytes or TH17 cells were distributed in 384-well plates (Optiplate; Perkin Elmer) (100,000 cells/well). Cells were pre-incubated for 30 minutes with different concentrations of peptide APG-2305 or APG-2309 and incubated with 25 ng/ml of IL-23 (R&D systems) for 10 minutes. STAT3 phosphorylation was determined with the kit Alpha Screen SureFire p-STAT-3 assay from Perkin Elmer according to manufacturer's instructions. Briefly, after incubation with IL-23, cells were lysed and contacted with (1) protein A acceptor conjugated beads coated with anti-p-STAT-3 (Tyr705) and (2) streptavidin-coated donor beads. The resulting signal was measured with a Perkin Elmer Wallac En vision 2104 Multilabel reader. Graphs were generated using GraphPrism 4 software.

### Example 3

### Derivatives of APG-2305 and APG-2309

The effect of peptide APG-2305 and APG-2309 derivatives on IL-23-induced STAT3 phosphorylation was determined using CD-1 mice freshly isolated splenocytes and the Alpha Screen p-STAT3 assay (see above materials and methods) (FIGS. 6 and 7). Both APG-2305 and APG-2309 peptides, and some of the derivatives, showed efficacy in inhibiting IL-23-induced STAT 3 phosphorylation in mice splenocytes and in pro-inflammatory TH17 cells where IL-23 has been shown to play major proliferative and anti-apoptotic roles. As described herein, based on the efficacy of derivatives, we have identified regions in APG-2305 and APG-2309 peptides that are important for their ability to affect IL-23R activity.

### Example 4

### In Vivo Efficacy of Peptide 2309

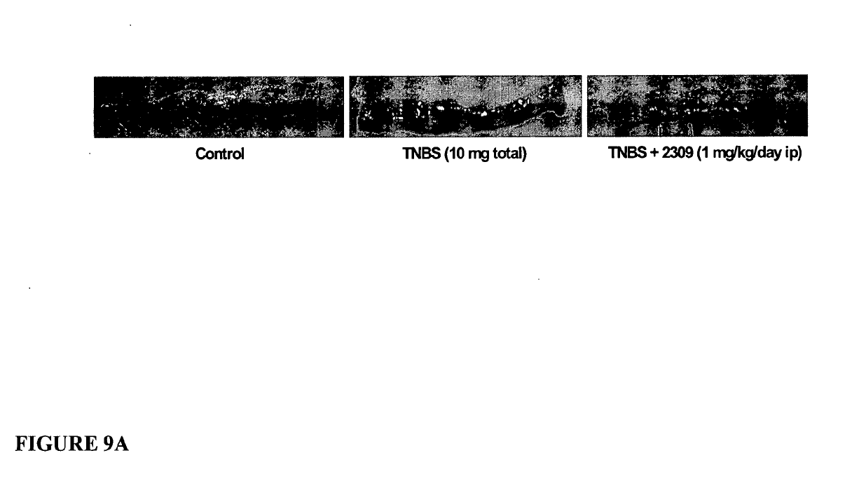

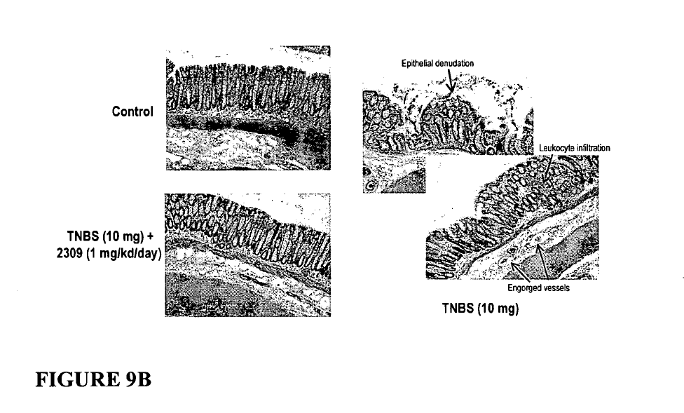

To test the efficacy of peptide 2309 in treating inflammatory bowel disease (IBD), a representative rat model was used. In the model, IBD induced by TNBS (trinitrobenzene sulfonic acid) as previously used to assess the role of IL-23 (Becker et al., J Immunol 177:2760-2764 (2006); Camoglio et al., Eur J Immunol 32:261-269 (2002); Zhang et al., Inflamm Bowel Dis 12:382-388 (2006)). Systemic administration of peptide 2309 (1 mg/kg/day, intraperitoneally; estimated tissue concentration 10 nM) abolished TNBS-induced intestinal inflammation, as detected by absence of inflammatory redness and edema on macroscopy (FIG. 9A), and essentially normal histology of intestinal mucosa and submucosa (FIG. 9B).

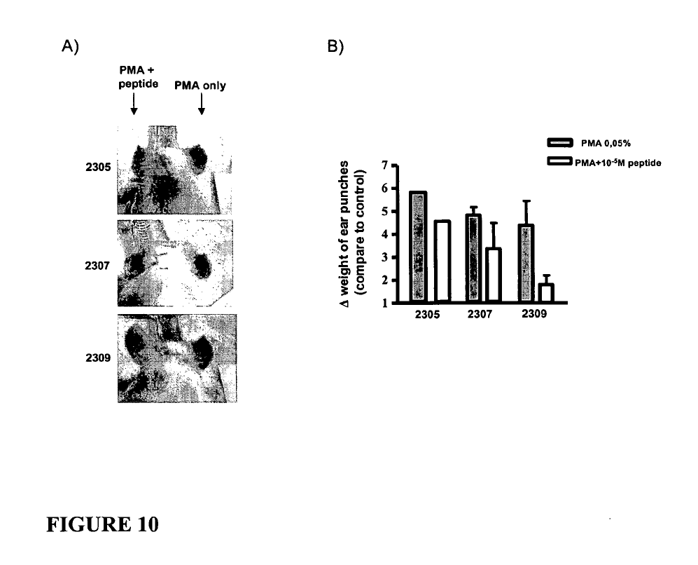

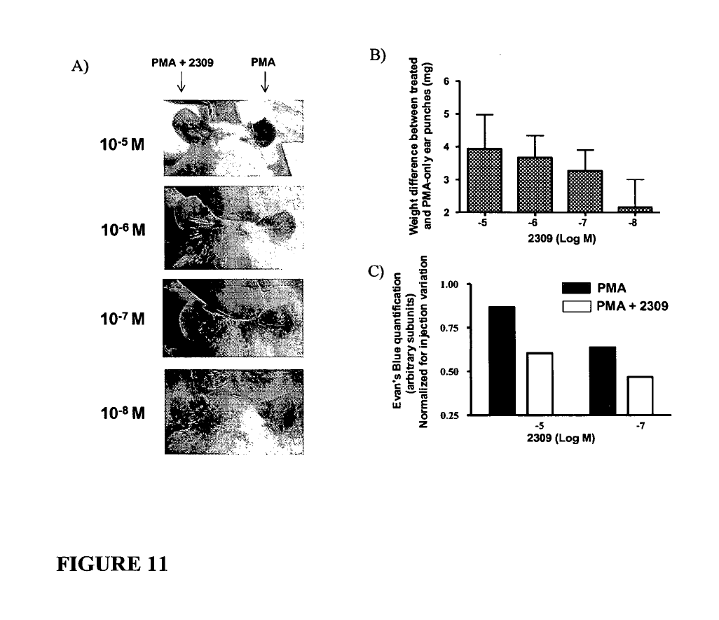

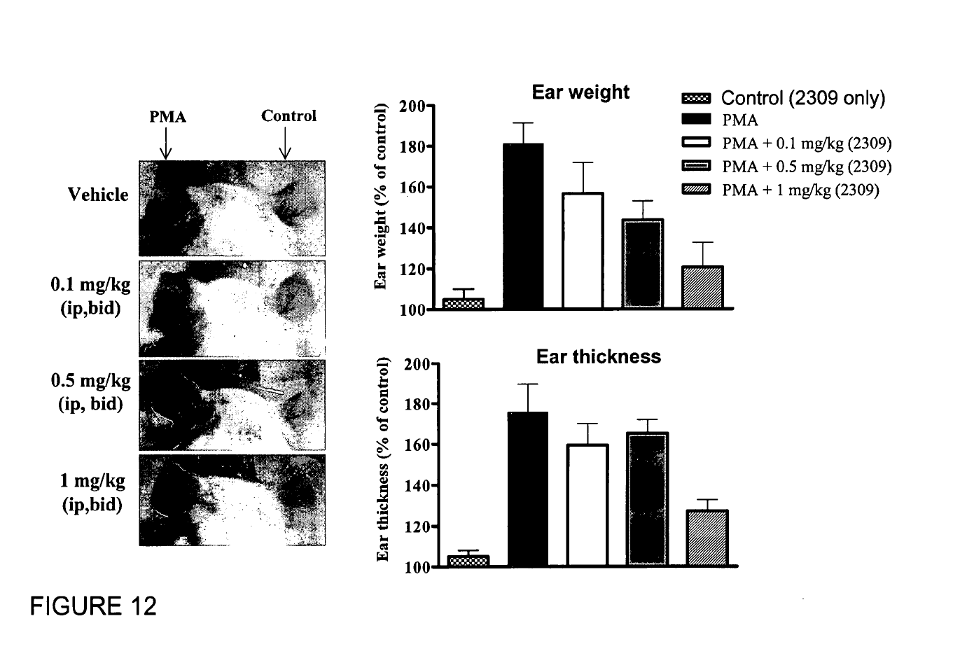

The efficacy of topically applied peptide 2305, 2307, or 2309 on PMA-induced dermatitis was determined (an art recognized a model of chronic and scaling dermatitis, which is suggestive of psoriatic dermatitis (Petersen et al., Basic & Clinical Pharm Tox 99:104-115 (2006); Schon, J Investig Derm 112:405-410 (1999)). Peptides 2305, 2307, or 2309 (final concentration: 5 μM) were mixed in polyethylene glycol (PEG-400) and 50 μl of the mixture was applied to the ear twice a day over a period of 3-5 days. Peptide 2309 caused a marked diminution in redness and ear weight (measure of edema); this effect was less pronounced with peptide 2307 (FIG. 10). Dose-response analysis revealed an EC₅₀≈50 nM of topical peptide 2309 in reducing two parameters of edema, namely weight and capillary leak (detected by Evans Blue extravasation) (FIG. 11). The results are indicative of effective transdermal penetration of peptide 2309. Further, intraperitoneal injection of peptide 2309 was also able to treat PMA-induced dermatitis as evidenced by a decrease in redness and ear weight (FIG. 12).

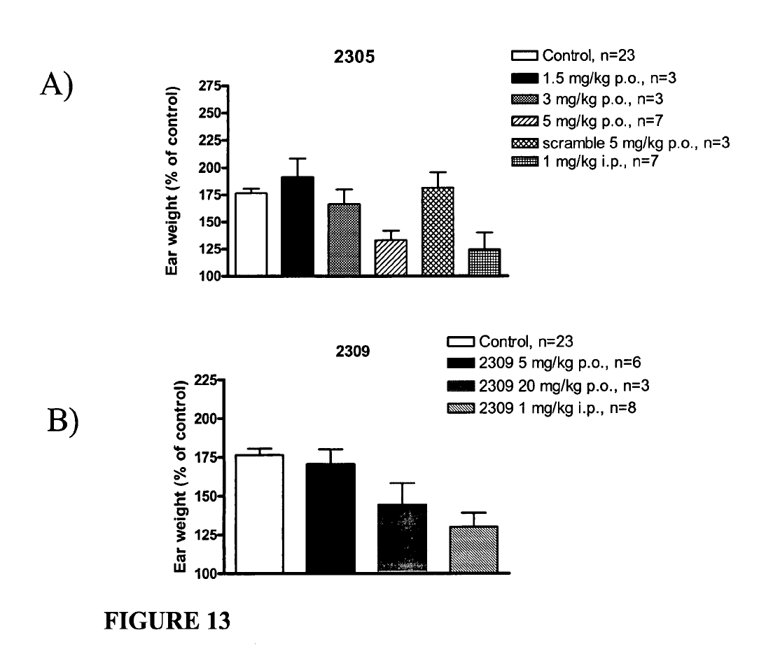

Peptides APG-2305 and APG-2309 were also given by enteral administration to rats to verify their oral availability. The peptides at various concentrations were injected (twice a day; 200 μl) into the stomach with a gavage needle (Number 20) starting at day 2 of PMA treatment. On day 4 the total peptide dose was given as a bolus and animals were sacrificed 4-6 hours later. As shown in FIG. 13A, APG-2305 injected enterally prevented ear edema in a dose-dependent fashion. A 5 mg/kg/day treatment resulted in total prevention of edema. Scrambled peptide of APG-2305 (5 mg/kg/day) and intraperitoneal APG-2305 served as negative and positive controls respectively. APG-2309 was also injected enterally but inhibit PMA-induced ear edema only slightly at 20 mg/kg administered orally (FIG. 13B). APG-2309 is more hydrophobic than APG-2305. These results indicate that peptides 2305 and, to a lesser extent, 2309 may be administered orally.

### Example 5

### Efficacy of APG-2309 in an Experimental Autoimmune Encephalitis Model

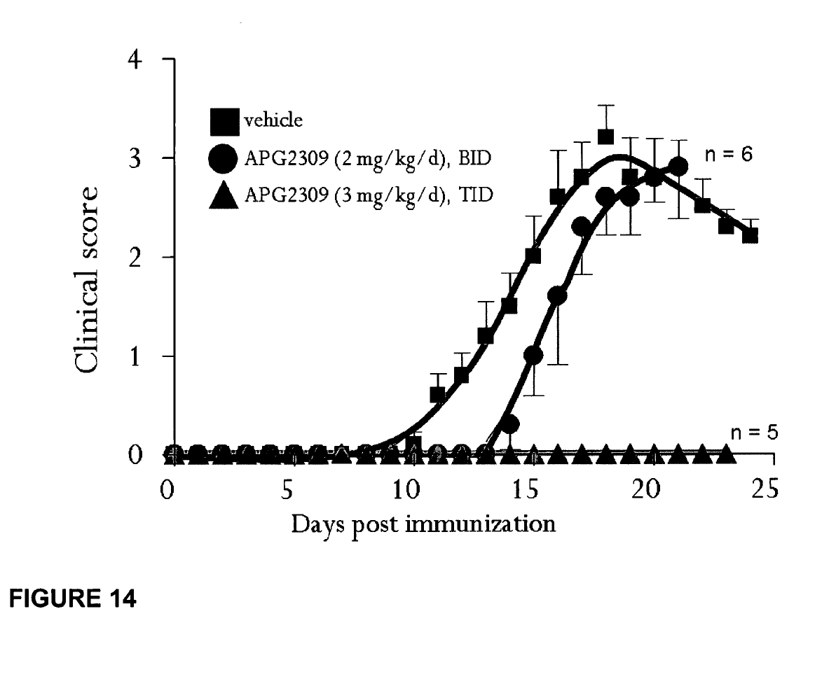

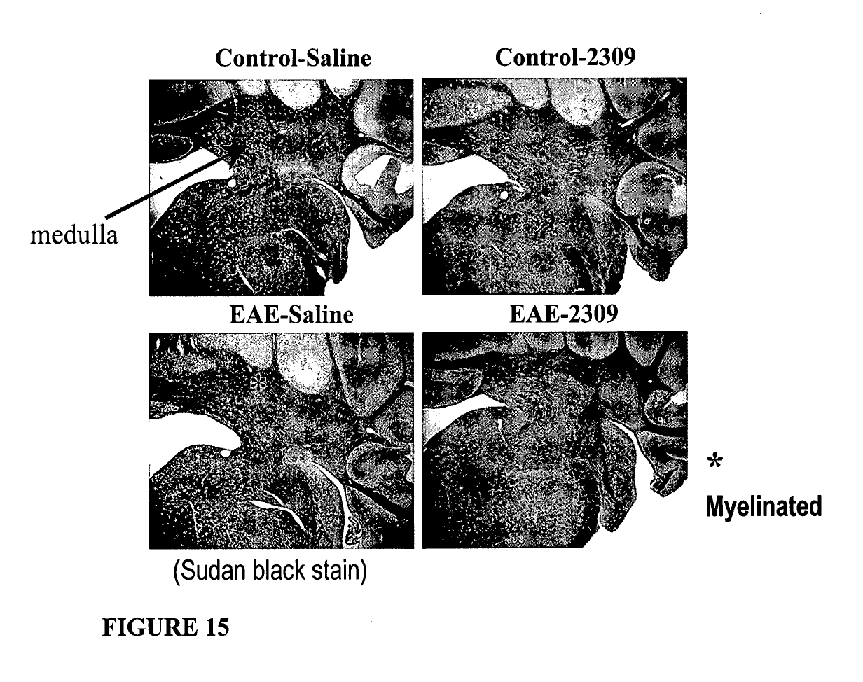

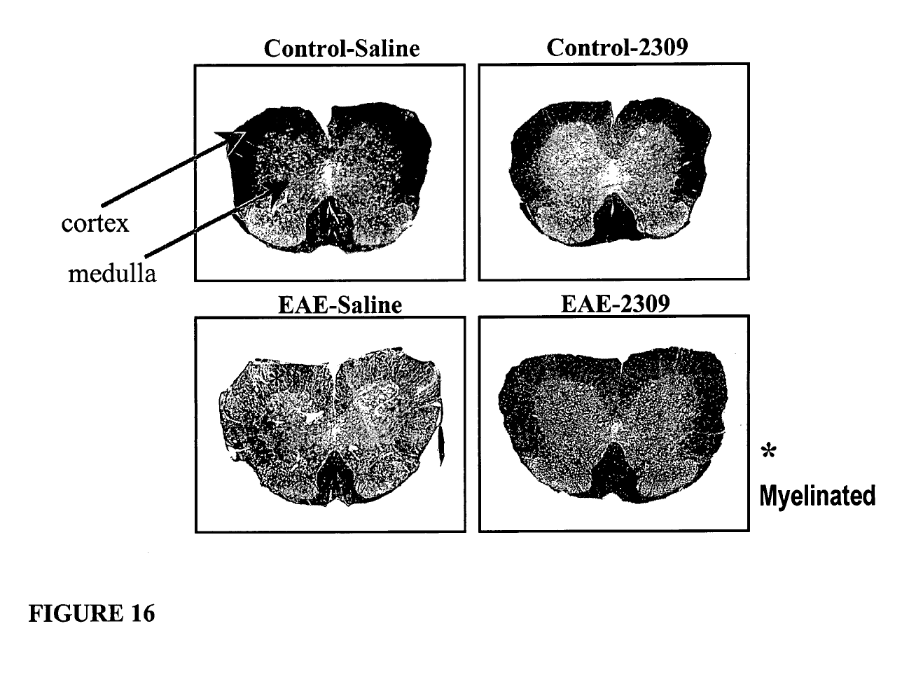

The Experimental Autoimmune Encephalitis (EAE) model of multiple sclerosis has been used to demonstrate the major involvement of IL-23 and not IL-12 in demyelinating disease. IL-23 is involved in the maintenance and survival of TH17 cells responsible for the secretion of major inflammatory molecules such as IL-22, IL-17, CCL-2, and CCL-5. The EAE model is the most used and currently most widely accepted animal model for multiple sclerosis. We tested the APG-2309 peptide in the EAE model. Clinical score evaluation was performed once a day. As is shown in FIG. 14, peptide 2309 retarded or completely abolished the clinical outcome of MOG-induced inflammation. While the administration of 2309 twice a day at 2 mg/kg/day retarded the appearance of the clinical signs of EAE in treated mice, a treatment three times a day completely abolished the induction of the disease symptoms. As is shown in FIGS. 15 and 16, the APG-2309 peptide totally prevents the demyelination of the cerebellum and the spinal cord.

The above experiments were carried out using the following materials and methods.

C57/BL6 mice were injected daily with the MOG peptide (MEVGWYRSPFSRVVHLYRNGK; SEQ ID NO:29) dissolved in 0.1 ml of saline with 4 mg/ml of Mycobacterium tuberculosis H37Ra and emulsified (1:1) in Freund adjuvant. Mice also received a pertussin toxin IV injection to permeabilise the blood-barrier membrane. Peptide injections were started 3 days before the MOG injection. Every day mice were weighed and scored clinically according to the following criterias:

Clinical score: 0=normal; 1=lost of tail tone; 2=flactonic tail; 3=incomplete paralysis of one or two hind limbs; 4=complete hind paralysis; 5=incomplete paralysis of 3 or 4 limbs; 6=complete paralysis of 3 limbs; 7=moribund; 8=dead. After three weeks of treatment, mice were sacrificed, and perfused with saline and a fixing solution (paraformaldehyde solution). Brains and spinal cords were harvested, frozen, cut into thin slices of 30 μm, and stained with Sudan Black stain (lipid staining) to visualize demyelinization.

Data were analyzed by 2-way analysis of variance factoring for drug treatment and dose, and followed by the comparison among means test. The best-fit curve was determined by the method of least squares for a polynomial analysis. Expecting (at least) a 65% difference in inflammatory parameters between control and treated groups (based on in vitro efficacy STAT3 phosphorylation and in vivo with β=0.2 and α=0.05) ˜9 animals were needed for each analysis factor (7-9 treatments, including control and 3 doses).

It is contemplated that any embodiment discussed in this specification can be implemented with respect to any method or composition of the invention, and vice versa. Furthermore, compositions and kits of the invention can be used to achieve methods of the invention.

Other objects, features and advantages of the present invention will become apparent to one skilled in the art based on the above detailed description. As such, it should be understood that the detailed description and the specific examples, while indicating specific embodiments of the invention, are given by way of illustration only, as various changes and modifications within the spirit and scope of the invention.

All patents, patent application publications, patent applications, and publications mentioned in this specification are herein incorporated by reference to the same extent as if each independent patent, patent application publication, patent application, or publication was specifically and individually indicated to be incorporated by reference.

What is claimed is:

1. A compound comprising a sequence characterized by the formula T₁E₂E₃E₄Q₅Q₆Y₇L₈, wherein said compound is 25 or fewer amino acids in length, wherein said compound antagonizes a biological activity of an interleukin 23 (IL-23) receptor, and wherein;
T₁ is selected from the group consisting of no residue, threonine, phenylalanine, alanine, and Σ, where Σ defines an alpha-amino acid comprising a hydrophobic side-chain Σ or aromatic side chain;
E₂, E₃, and E₄ each independently is selected from the group consisting of no residue, alanine, glutamic acid, glutamine, aspartic acid, asparagine, serine, histidine, homoserine, beta-leucine, beta-phenylalanine, alpha amino adipic acid, and Ψ where Ψ is a 3-amino-5-phenylpentanoic acid-alpha-amino acid comprising a hydrophobic side-chain, an aromatic amine, an aliphatic amine or a primary arylalkyl amine;
Q₅ and Q₆ each independently is selected from the group consisting of no residue, alanine, glutamine, aspartic acid, asparagine, glutamic acid, serine, homoserine, alpha-amino adipic acid, and Ψ where Ψ is a 3-amino-5-phenylpentanoic acid-alpha-amino acid comprising a hydrophobic side-chain, an aromatic amine, an aliphatic amine or a primary arylalkyl amine;
Y₇ is selected from the group consisting of no residue, tyrosine, phenylalanine, tryptophan, alanine, histidine, pyridylalanine, and Σ where Σ is an alpha-amino acid comprising a hydrophobic side-chain Σ or aromatic side chain or heteroaromatic side chain; and
L₈ is selected from the group consisting of no residue, leucine, alanine, valine, methionine, phenylalanine, tryptophan, and φ where φ is an alpha-amino acid comprising a hydrophobic side-chain; an aliphatic amine of one to ten carbons; or an aromatic or arylalkylamine; or a heteroaromatic or heteroarylalkylamine.

2. The compound of claim 1, wherein said compound comprises at least one D-amino acid.

3. The compound of claim 1, wherein said alpha-amino acid comprising a hydrophobic side-chain is nor-leucine, iso-leucine, tert-leucine, cyclohexylalanine, or allylglycine.

4. The compound of claim 1, wherein said aliphatic amine of one to ten carbons is a methylamine, iso-butylamine, iso-valerylamine, cyclopentylamine, cyclohexylmethylamine, or cyclohexylamine.

5-10. (canceled)

11. The compound of claim 1, wherein said compound consists of the sequence TEEEQQYL (SEQ ID NO:1), TAAEQQYL (SEQ ID NO:7), TAAAQQYL (SEQ ID NO:8), EEEQQYL (SEQ ID NO:9), AEEQQYL (SEQ ID NO:12), TEEEQ (SEQ ID NO:15), TEEE (SEQ ID NO:16), TEEEQAYL (SEQ ID NO:17), or TEEEAAYL (SEQ ID NO:18).

12. The compound of claim 11, wherein at least one amino acid is a D-amino acid.

13-17. (canceled)

18. A compound comprising a sequence characterized by the formula M₁E₂E₃S₄K₅Q₆L₇Q₈L₉, wherein said compound is 25 or fewer amino acids in length, wherein said compound antagonizes a biological activity of an interleukin 23 (IL-23) receptor, and wherein;
M₁ is selected from the group consisting of no residue, methionine, valine, leucine, alanine, phenylalanine, tryptophan, and φ where φ is an alpha-amino acid comprising a hydrophobic side-chain; an aliphatic amine of one to ten carbons; or an aromatic or arylalkylamine;
E₂ and E₃ each independently is selected from the group consisting of no residue, alanine, glutamic acid, glutamine, aspartic acid, asparagine, serine, histidine, homoserine, beta-leucine, beta-phenylalanine, alpha amino adipic acid, trimesic acid, or an alpha,omega-dicarboxylic acid (e.g., succinic acid, glutaric acid, or azelic acid), and Ψ where Ψ is a 3-amino-5-phenylpentanoic acid-alpha-amino acid comprising a hydrophobic side-chain, an aromatic amine, an aliphatic amine or a primary arylalkyl amine;
S₄ is selected from the group consisting of no residue, serine, threonine, allothreonine, hydroxyproline, beta-hydroxyvaline, valine, and η, where η is a neutral hydrophilic amino acid;
K₅ is selected from the group consisting of no residue, glutamine, lysine, arginine, histidine, alanine, ornithine, citruline, 2-pyridylalanine, 3-pyridylalanine, 4-pyridylalanine, and an arginine surrogate;
Q₆ is selected from the group consisting of no residue, alanine, glutamine, aspartic acid, asparagine, glutamic acid, serine, and Ψ where Ψ is a 3-amino-5-phenylpentanoic acid-alpha-amino acid comprising a hydrophobic side-chain, an aromatic amine, an aliphatic amine or a primary arylalkyl amine;
L₇ is selected from the group consisting of no residue, leucine, alanine, valine, methionine, phenylalanine, tryptophan, and φ where φ is an alpha-amino acid comprising a hydrophobic side-chain; an aliphatic amine of one to ten carbons; or an aromatic or arylalkylamine; or a heteroaromatic or heteroarylalkylamine;
Q₈ is selected from the group consisting of no residue, glutamine, aspartic acid, asparagine, glutamic acid, serine, and Ψ where Ψ is a 3-amino-5-phenylpentanoic acid-alpha-amino acid comprising a hydrophobic side-chain, an aromatic amine, an aliphatic amine or a primary arylalkyl amine; and
L₉ is selected from the group consisting of no residue, leucine, isoleucine, alanine, valine, methionine, phenylalanine, tryptophan, and φ where φ is an alpha-amino acid comprising a hydrophobic side-chain; an aliphatic amine of one to ten carbons; or an aromatic or arylalkylamine; or a heteroaromatic or heteroarylalkylamine.

19. The compound of claim 18, wherein said compound comprises at least one D-amino acid.

20-26. (canceled)

27. The compound of claim 18, wherein said compound consists of the sequence MEESKQLQL (SEQ ID NO:2), MAESKQLQL (SEQ ID NO:19), MAASKQLQL (SEQ ID NO:20), ESKQLQL (SEQ ID NO:21), MEESKQLQI (SEQ ID NO:22), MEESKQL (SEQ ID NO:23), MEESKQ (SEQ ID NO:24), MEESQQLQI (SEQ ID NO:25), EESKQLQL (SEQ ID NO:26), VQAANALGMEESKQLQLHLDDLVL (SEQ ID NO:27), or LVLDDLHLQLQKSEEMGLANAAQV (SEQ ID NO:28).

28. The compound of claim 27, wherein at least one amino acid is a D-amino acid.

29-32. (canceled)

33. A compound comprising a sequence characterized by the formula K₁K₂Y₃L₄V₅W₆V₇Q₈, wherein said compound is 25 or fewer amino acid in length, wherein said compound antagonizes a biological activity of an interleukin 23 (IL-23) receptor, and wherein;
K₁ and K₂ each independently is selected from the group consisting of no residue, lysine, arginine, histidine, alanine, ornithine, citruline, 2-pyridylalanine, 3-pyridylalanine, 4-pyridylalanine, and an arginine surrogate;
Y₃ is selected from the group consisting of no residue, tyrosine, phenylalanine, tryptophan, alanine, and Σ where Σ is an alpha-amino acid comprising a hydrophobic side-chain Σ or aromatic side chain, or heteroaromatic side chain;
L₄ is selected from the group consisting of no residue, leucine, alanine, valine, methionine, phenylalanine, tryptophan, and φ where φ is an alpha-amino acid comprising a hydrophobic side-chain; an aliphatic amine of one to ten carbons; or an aromatic or arylalkylamine; or a heteroaromatic or heteroarylalkylamine;
V₅ is selected from the group consisting of no residue, valine, leucine, alanine, methionine, phenylalanine, tryptophan, and φ where φ is an alpha-amino acid comprising a hydrophobic side-chain; an aliphatic amine of one to ten carbons; or an aromatic or arylalkylamine; or a heteroaromatic or heteroarylalkylamine;
W₆ is selected from the group consisting of no residue, tryptophan, tyrosine, phenylalanine, alanine, and Σ where Σ is an alpha-amino acid comprising a hydrophobic side-chain Σ or aromatic side chain; or heteroaromatic side chain;
V₇ is selected from the group consisting of no residue, valine, leucine, alanine, methionine, phenylalanine, tryptophan, and φ where φ is an alpha-amino acid comprising a hydrophobic side-chain; an aliphatic amine of one to ten carbons; or an aromatic or arylalkylamine; or a heteroaromatic or heteroarylalkylamine; and
Q₈ is selected from the group consisting of no residue, glutamine, aspartic acid, asparagine, glutamic acid, serine, and Ψ where Ψ is a 3-amino-5-phenylpentanoic acid-alpha-amino acid comprising a hydrophobic side-chain, an aromatic amine, an aliphatic amine or a primary arylalkyl amine.

34. The compound of claim 33, wherein said compound comprises at least one D-amino acid.

35-47. (canceled)

48. A compound comprising a sequence characterized by the formula L₁P₂D₃E₄V₅T₆C₇V₈, wherein said compound is 25 or fewer amino acids in length, wherein said compound antagonizes a biological activity of an interleukin 23 (IL-23) receptor, and wherein;
L₁ is selected from the group consisting of no residue, leucine, alanine, valine, methionine, phenylalanine, tryptophan, and φ where φ is an alpha-amino acid comprising a hydrophobic side-chain; an aliphatic amine of one to ten carbons; or an aromatic or arylalkylamine;
P₂ is selected from the group consisting of no residue, proline, alanine, N-methylglycine, N-isobutylglycine, aminoisobutyric acid (Aib), N-Methyl-L-alanine (MeAla), trans-4-Hydroxyproline, diethylthiazolidine carboxylic acid (Dtc), and Ω, where Ω is a conformational constraint-producing amino acid;
D₃ is selected from the group consisting of no residue, aspartic acid, asparagine, glutamic acid, glutamine, serine, histidine, homoserine, beta-leucine, beta-phenylalanine, alpha amino adipic acid, and Ψ where Ψ is a 3-amino-5-phenylpentanoic acid-alpha-amino acid comprising a hydrophobic side-chain, an aromatic amine, an aliphatic amine, or a primary arylalkyl amine;
E₄ is selected from the group consisting of no residue, alanine, glutamic acid, glutamine, aspartic acid, asparagine, serine, histidine, homoserine, beta-leucine, beta-phenylalanine, alpha amino adipic acid, and Ψ where Ψ is a 3-amino-5-phenylpentanoic acid-alpha-amino acid comprising a hydrophobic side-chain, an aromatic amine, an aliphatic amine or a primary arylalkyl amine;
V₅ is selected from the group consisting of no residue, valine, leucine, alanine, methionine, phenylalanine, tryptophan, and φ where φ is an alpha-amino acid comprising a hydrophobic side-chain; an aliphatic amine of one to ten carbons; or an aromatic or arylalkylamine; or a heteroaromatic or heteroarylalkylamine;
T₆ is selected from the group consisting of no residue, threonine, phenylalanine, alanine, and Σ where Σ defines an alpha-amino acid comprising a hydrophobic side-chain Σ or aromatic side chain;
C₇ is selected from the group consisting of no residue, cysteine, serine, homoserine, homocysteine, threonine, methionine, N-acetylcysteine, cystathionine, 2-aminobutyrate, and β,β-dimethylcysteine (Penicillamine); and
V₈ is selected from the group consisting of no residue, valine, leucine, alanine, methionine, phenylalanine, tryptophan, and φ where φ is an alpha-amino acid comprising a hydrophobic side-chain; an aliphatic amine of one to ten carbons; or an aromatic or arylalkylamine.

49. The compound of claim 48 wherein said compound comprises at least one D-amino acid.

50-62. (canceled)

63. A vector comprising an isolated nucleic acid sequence encoding the amino acid sequence of any one of SEQ ID NOS:1-5.

64. A cell comprising the vector of claim 63.

65-66. (canceled)

67. A cell expressing the compound of claim 1.

68-69. (canceled)

70. A pharmaceutical composition comprising the compound of claim 1.

71. A method of treating an autoimmune or inflammatory disorder, said method comprising administering to a subject in need thereof an effective dose of the compound of claim 1.

72-74. (canceled)

75. A method of identifying a candidate compound that inhibits or enhances the ability of the compound of claim 1 to antagonize a biological activity of an interleukin 23 receptor, said method comprising:
(i) contacting the interleukin 23 receptor with the candidate compound in the presence of the compound of claim 1; and
(ii) assaying for an increase or decrease of the biological activity of the interleukin 23 receptor relative to a control not contacted with the candidate compound, wherein a decrease of said biological activity relative to said control indicates that said candidate compound enhances the ability of the compound of claim 1 to antagonize a biological activity of an interleukin 23 receptor, and wherein an increase of said biological activity relative to said control indicates that said candidate compound inhibits the ability of the compound of claim 1 to antagonize a biological activity of an interleukin 23 receptor.

76-79. (canceled)
